SEQUENCE LISTING (17 sequences)

SEQ ID NO: 1 - DNA, 48 bp, Artificial Sequence
  Description of Artificial Sequence Synthetic oligonucleotide
aacaccgggaggatagttcggtggctgttcagggtctcctcccggtga

SEQ ID NO: 2 - DNA, 81 bp, Artificial Sequence
  Description of Artificial Sequence Synthetic oligonucleotide
atccagagtgacgcagcagatcagtctatcttctcctgatgggttcctagttataggtgaagctggacacggtggcttagt

SEQ ID NO: 3 - DNA, 54 bp, Artificial Sequence
  Description of Artificial Sequence Synthetic oligonucleotide
acagcagatcagtctatcttctcctgatgggttcctatttataggtgaagctgt

SEQ ID NO: 4 - DNA, 63 bp, Artificial Sequence
  Description of Artificial Sequence Synthetic oligonucleotide
aacaccgggaggatagttcggtggctgttcagggtctcctcccggtgattttttttttttttt

SEQ ID NO: 5 - DNA, 58 bp, Artificial Sequence
  Description of Artificial Sequence Synthetic oligonucleotide
aacaccgggaggatagttcggtggctgttcagggtctcctcccggtgatttttttttt

SEQ ID NO: 6 - DNA, 51 bp, Artificial Sequence
  Description of Artificial Sequence Synthetic oligonucleotide
atctaactgctgcgccgccgggaaaatactgtacggttagatttttttttt

SEQ ID NO: 7 - DNA, 48 bp, Artificial Sequence
  Description of Artificial Sequence Synthetic oligonucleotide
nnnnnnnnnnnnnnnnnnnnnnnnnnnnnnnnnnnnnnnnnnnnnnnn

SEQ ID NO: 8 - DNA, 48 bp, Artificial Sequence; identical to SEQ ID NO: 1
  Description of Artificial Sequence Synthetic oligonucleotide

SEQ ID NO: 9 - DNA, 48 bp, Artificial Sequence; identical to SEQ ID NO: 1
  Description of Artificial Sequence Synthetic oligonucleotide

SEQ ID NO: 10 - DNA, 48 bp, Artificial Sequence; identical to SEQ ID NO: 1
  Description of Artificial Sequence Synthetic oligonucleotide

SEQ ID NO: 11 - DNA, 81 bp, Artificial Sequence; identical to SEQ ID NO: 2
  Description of Artificial Sequence Synthetic oligonucleotide

SEQ ID NO: 12 - DNA, 54 bp, Artificial Sequence; identical to SEQ ID NO: 3
  Description of Artificial Sequence Synthetic oligonucleotide

SEQ ID NO: 13 - DNA, 100 bp, Artificial Sequence
  Description of Artificial Sequence Synthetic polynucleotide
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 14 - DNA, 48 bp, Artificial Sequence
  Description of Artificial Sequence Synthetic oligonucleotide
tttttttatctaactgctgcgccgccgggaaaatactgtacggttaga

SEQ ID NO: 15 - DNA, 48 bp, Artificial Sequence; identical to SEQ ID NO: 1
  Description of Artificial Sequence Synthetic oligonucleotide

SEQ ID NO: 16 - DNA, 48 bp, Artificial Sequence; identical to SEQ ID NO: 7
  Description of Artificial Sequence Synthetic oligonucleotide

SEQ ID NO: 17 - DNA, 63 bp, Artificial Sequence
  Description of Artificial Sequence Synthetic oligonucleotide
nnnnnnnnnnnnnnnnnnnnnnnnnnnnnnnnnnnnnnnnnnnnnnnnttttttttttttttt

(19) United States
(12) Patent Application Publication
(10) Pub. No.: US 2012/0087949 A1
(43) Pub. Date: Apr. 12, 2012

(54) OLIGONUCLEOTIDE MICELLES

(75) Inventors: Weihong Tan, Gainesville, FL (US); Liu Haipeng, Malden, MA (US); Yanrong Wu, Greensboro, NC (US)

(73) Assignee: University of Florida Research Foundation, Inc., Gainesville, FL (US)

(21) Appl. No.: 13/141,027

(22) PCT Filed: Dec. 18, 2009
(86) PCT No.: PCT/US09/68864

§ 371 Date: Dec. 7, 2011

Related U.S. Application Data
(60) Provisional application No. 61/203,193, filed on Dec. 19, 2008.

(51) Int. Cl. A61K 31/7088; C12Q 1/02; C12N 5/00; A61K 9/00

(52) U.S. Cl. 424/400; 514/44 R; 514/44 A; 435/29; 435/375

(57) ABSTRACT

The present invention provides homogenous populations of micelles, methods for preparing these populations, methods for treating or preventing a disease or disorder using the population of micelles of the invention.

### CROSS-REFERENCE TO RELATED APPLICATION

This application claims the benefit of U.S. Provisional Application No. 61/203,193, filed on Dec. 12, 2008, the entire contents of each of which are incorporated herein by reference.

### BACKGROUND OF THE INVENTION

Amphiphilic compounds are compounds with hydrophilic and hydrophobic regions. Amphiphilic peptides, nucleic acids, lipids and polymers are able to generate ordered supramolecular structures such as monolayers, micelles, vesicles, bilayers and nanotubes. When dispersed in water at a concentration above their critical micelle concentration (CMC), amphiphilic compounds spontaneously self-associate into micelles. For biomedical applications, micelle structures are of particular interest because of their small size, good biocompatibility, high stability both in vitro and in vivo, and the ability to transport poorly soluble pharmaceuticals.

While a variety of micelle systems have been demonstrated, the design and construction of self-assembled micelle systems for example, for therapeutic or for nanobiotechnological applications, requires considerable attention to the molecular engineering of the corresponding building blocks, since all the information required for the assembly must be encoded in their molecular architecture.

The ability to control the micelle size in a pharmaceutical formulation has been limited, and thus the control of the rate of delivery of a drug to a site of action is limited due to the inability to control the size of the micelle in solution. As an important solubilizing agent, self-assembled micelles are a key component of pharmaceutical and biological applications. However, their efficacy could be improved by controlling the self-assembly dynamics. This would require an engineering design capable of producing the formation of monodispersed functional micelles characterized by beneficial changes in size, shape and chemical structure.

Thus, there remains a need in the art to provide controlled or homogenous populations of micelles.

### SUMMARY

The instant invention describes the design, construction and assembly, e.g. self-assembly of a population of uniform and homogenous amphiphilic oligonucleotide micelles. The invention is based on the finding that in aqueous solutions, the amphiphilic molecules spontaneously self-assemble into monodispersed micellar structures with high thermodynamic stability and can be disassembled for insertion into a model membrane. The present invention provides homogenous populations of micelles, and methods for preparing these populations.

In a first aspect, the invention is directed to a population of micelles comprising oligonucleotides and hydrophobic lipid elements, wherein the population is homogenous.

In another aspect, the invention features a pharmaceutical composition comprising a population of micelles comprising oligonucleotides and hydrophobic lipid elements, wherein the population is homogenous, and a pharmaceutically acceptable carrier.

In one embodiment of any of the above aspects, the population is homogenous in diameter. In another embodiment, the population is homogenous in weight.

In a further embodiment of any of the above aspects, the hydrophobic lipid elements form a hydrophobic core.

In another embodiment of any of the above aspects, the oligonucleotides comprise single stranded DNA. In a related embodiment, the single stranded DNA is between 2-100 nucleotide bases in length. In a further embodiment of any of the above aspects, the single stranded DNA is 5 nucleotide bases in length. In another embodiment of any of the above aspects, the single stranded DNA is 20 nucleotide bases in length. In another further embodiment of any of the above aspects, the single stranded DNA is 50 nucleotide bases in length.

In one embodiment of any of the above aspects, the oligonucleotides and hydrophobic elements are linked by a covalent bond. In a particular embodiment, the covalent bond is a non-cleavable linkage. In another particular embodiment, the covalent bond is a cleavable linkage.

In another embodiment of any of the above aspects, the population of micelles further comprises reporter molecules. In a related embodiment, the reporter molecules are fluorescent. In a further related embodiment, the reporter molecules are pyrene molecules.

In one embodiment of any of the above aspects, the hydrophobic elements are selected from linear, branched chain and cyclic hydrophobic elements. In another embodiment of any of the above aspects, the hydrophobic element is between 8 and 25 units in length. In a particular embodiment of any of the above aspects, the hydrophobic element is 18 units in length. In another embodiment of any of the above aspects, the chain elements of said hydrophobic element are carbon atoms.

In one embodiment of any of the above aspects, the oligonucleotide is selected from an antisense oligonucleotide, a decoy oligonucleotide, a siRNA, a DNAzyme, a ribozyme, and an aptamer. In another embodiment of any of the above aspects, the oligonucleotides comprise naturally occurring or modified nucleosides such as DNA, RNA, locked nucleic acids (LNA's), 2′O-methyl RNA (2′Ome), 2′O-methoxyethyl RNA (2′MOE) in their phosphate or phosphothioate forms or Morpholinos or peptide nucleic acids (PNA's).

In another embodiment of any of the above aspects, the micelles have a mean diameter of 3-50 nm in an aqueous solution.

In another embodiment of any of the above aspects, the population has a molecular weight ranging from 1,000 to 50,000.

In a related embodiment of any of the above aspects, the micelles further comprise an agent. In a further related embodiment of any of the above aspects, the agent is loaded into the hydrophobic core. In still another embodiment of any of the above aspects, the agent is a therapeutic agent. In a further related embodiment of any of the above aspects, the therapeutic agent is selected from a drug, a toxin, a gene, a small molecule and an oligonucleotide.

In another embodiment of any of the above aspects, the agent is an imaging agent.

In one embodiment of any of the above aspects, the oligonucleotide is selected from an antisense oligonucleotide, a decoy oligonucleotide, a siRNA, a DNAzyme, a ribozyme, and an aptamer.

In one embodiment of any one of the above aspects, the oligonucleotide is an aptamer.

In another embodiment of any one of the above aspects, the aptamer is selected from SEQ ID NO: 1 (AAC ACC GGG AGG ATA GTT CGG TGG CTG TTC AGG GTC TCC TCC CGG TGA), SEQ ID NO: 2 (ATC CAG AGT GAC GCA GCA GAT CAG TCT ATC TTC TCC TGA TGG GTT CCT AGT TAT AGG TGA AGC TGG ACA CGG TGG CTT AGT), SEQ ID NO: 3 (ACA GCA GAT CAG TCT ATC TTC TCC TGA TGG GTT CCT ATT TAT AGG TGA AGC TGT, SEQ ID NO: 4 (AAC ACC GGG AGG ATA GTT CGG TGG CTG TTC AGG GTC TCC TCC CGG TGA TTT TTT TTT TTT TTT), SEQ ID NO: 5(5′-AAC ACC GGG AGG ATA GTT CGG TGG CTG TTC AGG GTC TCC TCC CGG TGA TTT TTT TTT T-biotin-3), SEQ ID NO: 6 (5′-ATC TAA CTG CTG CGC CGC CGG GAA AAT ACT GTA CGG TTA GAT TTT TTT TTT-biotin-3)′.

In another embodiment of any one of the above aspects, the micelle is selected from SEQ ID NO: 8 (5′-lipid tail-(CH2CH2O)24-AAC ACC GGG AGG ATA GTT CGG TGG CTG TTC AGG GTC TCC TCC CGG TGA-FAM-3′, SEQ ID NO: 9 (5′-lipid tail-(CH2CH2O)24-AAC ACC GGG AGG ATA GTT CGG TGG CTG TTC AGG GTC TCC TCC CGG TGA-biotin-3′, SEQ ID NO: 10 (5′-lipid tail-(CH2CH2O)24-AAC ACC GGG AGG ATA GTT CGG TGG CTG TTC AGG GTC TCC TCC CGG TGA-TMR-3′), SEQ ID NO: 11 (5′-lipid tail-(CH2CH2O)24-ATC CAG AGT GAC GCA GCA GAT CAG TCT ATC TTC TCC TGA TGG GTT CCT AGT TAT AGG TGA AGC TGG ACA CGG TGG CTT AGT-FAM-3′), SEQ ID NO: 12 (5′-lipid tail-(CH2CH2O)24-ACA GCA GAT CAG TCT ATC TTC TCC TGA TGG GTT CCT ATT TAT AGG TGA AGC TGT-FAM-3′, SEQ ID NO: 13(5′-lipid tail-(CH2CH20)24-(N)n*-FAM-3′.

In a further embodiment of any of the above aspects, the drug is a hydrophobic drug.

In one embodiment of any of the above aspects, the oligonucleotide decreases expression of a target gene.

In another aspect, the invention features a method of preparing a homogenous population of micelles comprising preparing oligonucleotides, preparing hydrophobic lipid elements, and mixing oligonucleotides and hydrophobic lipid elements in an aqueous medium, thereby forming a homogenous population of micelles.

In another aspect, the invention features a method of preparing a homogenous population of micelles comprising preparing oligonucleotides, preparing hydrophobic lipid elements, preparing pyrene molecules; and mixing oligonucleotides, hydrophobic lipid elements and pyrene molecules in an aqueous medium, thereby forming a homogenous population of micelles.

In one embodiment of the above aspects, the invention further comprises mixing reporter molecules. In a related embodiment, the reporter molecules are fluorescent. In a further related embodiment, the reporter molecules are pyrene molecules.

In another embodiment of the above aspects, the aqueous medium is selected from water or saline.

In still another embodiment of the above aspects, the method further comprises mixing in an agent. In a related embodiment, the agent is loaded into the hydrophobic core. In a further related embodiment, the agent is a therapeutic agent.

In another related embodiment, the therapeutic agent is selected from a drug, a toxin, a gene, a small molecule and an oligonucleotide. In still another related embodiment, the oligonucleotide is selected from the group consisting of: an antisense oligonucleotide, a decoy oligonucleotide, a siRNA, a DNAzyme, a ribozyme, and an aptamer.

In one embodiment of the above aspects, the agent is an imaging agent.

In another embodiment, the invention features a population of micelles prepared by the any one of the methods as described herein.

In another embodiment, the invention features a method of delivering an oligonucleotide to a target cell comprising contacting the target cell with a population of micelles of any one of the aspects as described herein.

In another embodiment, the invention features a method of delivering an oligonucleotide to a subject comprising administering to the subject the population of micelles of any one of the aspects as described herein.

In another embodiment, the invention features a method of delivering nucleic acids to a target cell comprising contacting the target cell with a population of micelles of any one of the aspects as described herein.

In another embodiment, the invention features a method of delivering nucleic acids to a subject comprising administering to the subject the population of micelles of any one of the aspects as described herein.

In one embodiment, the oligonucleotide is selected from the group consisting of an antisense oligonucleotide, a decoy oligonucleotide, a siRNA, a DNAzyme, a ribozyme, and an aptamer.

In another embodiment, delivery is monitored by fluorescent signal.

In another embodiment, the invention features a method for treating a disease or disorder in a subject comprising administering to the subject the population of micelles of any one of the aspects as described herein.

In another aspect, the invention features a method of delivering an oligonucleotide to a target cell comprising contacting the target cell with a population of micelles comprising oligonucleotides and hydrophobic lipid elements; and incubating the target cell with the population of micelles, thereby delivering an oligonucleotide to the target cell.

In another aspect, the invention features a method of delivering an oligonucleotide to a subject comprising administering to the subject a population of micelles comprising oligonucleotides and hydrophobic lipid elements, thereby delivering an oligonucleotide to a subject.

In one embodiment, the oligonucleotide is a therapeutic oligonucleotide.

In another aspect, the invention features a method of delivering nucleic acids to a target cell comprising contacting the target cell with a population of micelles comprising nucleic acids and hydrophobic lipid elements, and incubating the target cell with the population of micelles, thereby delivering nucleic acids to the target cell.

In yet another aspect, the invention features a method of delivering nucleic acids to a subject comprising administering to the subject a population of micelles comprising nucleic acids and hydrophobic lipid elements and thereby delivering nucleic acids to the subject.

In still another aspect, the invention features a method for treating a disease or disorder in a subject comprising administering to the subject a population of micelles comprising oligonucleotides and hydrophobic lipid elements, thereby treating the disease or disorder in the subject.

In one embodiment, the disease or disorder is cancer.

In another embodiment, the therapeutic agent is selected from a drug, a toxin, a gene, a small molecule and an oligonucleotide. In still another related embodiment, the oligonucleotide is selected from the group consisting of: an antisense oligonucleotide, a decoy oligonucleotide, a siRNA, a DNAzyme, a ribozyme, and an aptamer.

In one embodiment of any one of the above aspects, the oligonucleotide is an aptamer.

In another embodiment of any one of the above aspects, the aptamer is selected from SEQ ID NO: 1 (AAC ACC GGG AGG ATA GTT CGG TGG CTG TTC AGG GTC TCC TCC CGG TGA), SEQ ID NO: 2 (ATC CAG AGT GAC GCA GCA GAT CAG TCT ATC TTC TCC TGA TGG GTT CCT AGT TAT AGG TGA AGC TGG ACA CGG TGG CTT AGT), SEQ ID NO: 3 (ACA GCA GAT CAG TCT ATC TTC TCC TGA TGG GTT CCT ATT TAT AGG TGA AGC TGT, SEQ ID NO: 4 (AAC ACC GGG AGG ATA GTT CGG TGG CTG TTC AGG GTC TCC TCC CGG TGA TTT TTT TTT TTT TTT), SEQ ID NO: 5(5′-AAC ACC GGG AGG ATA GTT CGG TGG CTG TTC AGG GTC TCC TCC CGG TGA TTT TTT TTT T-biotin-3), SEQ ID NO: 6 (5′-ATC TAA CTG CTG CGC CGC CGG GAA AAT ACT GTA CGG TTA GAT TTT TTT TTT-biotin-3)′.

In another embodiment of any one of the above aspects, the micelle is selected from SEQ ID NO: 8 (5′-lipid tail-(CH2CH2O)24-AAC ACC GGG AGG ATA GTT CGG TGG CTG TTC AGG GTC TCC TCC CGG TGA-FAM-3′, SEQ ID NO: 9 (5′-lipid tail-(CH2CH2O)24-AAC ACC GGG AGG ATA GTT CGG TGG CTG TTC AGG GTC TCC TCC CGG TGA-biotin-3′, SEQ ID NO: 10 (5′-lipid tail-(CH2CH2O)24-AAC ACC GGG AGG ATA GTT CGG TGG CTG TTC AGG GTC TCC TCC CGG TGA-TMR-3′), SEQ ID NO: 11 (5′-lipid tail-(CH2CH2O)24-ATC CAG AGT GAC GCA GCA GAT CAG TCT ATC TTC TCC TGA TGG GTT CCT AGT TAT AGG TGA AGC TGG ACA CGG TGG CTT AGT-FAM-3′), SEQ ID NO: 12 (5′-lipid tail-(CH2CH2O)24-ACA GCA GAT CAG TCT ATC TTC TCC TGA TGG GTT CCT ATT TAT AGG TGA AGC TGT-FAM-3′, SEQ ID NO: 13(5′-lipid tail-(CH2CH20)24-(N)n*-FAM-3′.

In another embodiment of any of the above aspects, the population of micelles further comprises reporter molecules. In a related embodiment, the reporter molecules are fluorescent. In a further related embodiment, the reporter molecules are pyrene molecules.

In another embodiment of any one of the above aspects, the method further comprises the step of monitoring delivery.

In yet another embodiment of any one of the above aspects, the target cell is located in a tumor.

In another embodiment of any one of the above aspects, the subject is suffering from a disease or disorder. In a related embodiment, the disease or disorder is cancer.

In another embodiment of any one of the above aspects, the population of micelles is administered to the subject orally or intravenously.

In another aspect, the invention features a kit comprising oligonucleotides and hydrophobic lipid elements, and instructions for use.

In one embodiment, the kit further comprises reporter molecules. In a related embodiment, the reporter molecules are fluorescent. In a further related embodiment, the reporter molecules are pyrene molecules.

Other aspects of the invention are described infra.

### BRIEF DESCRIPTION OF THE FIGURES

FIG. 1 (A and B) shows in (A) a schematic of the design and assembly of DNA micelles and containing a DNA corona, a pyrene unit (fluorescence reporter) and a lipid molecule. (B) Molecular structure of pyrene unit and lipid.

FIG. 2 (A-D) is four panels. Panel (A) shows fluorescence spectra analysis of the assembled micelles. Panel (B) shows photographic image of micelle aggregation in different solvent systems. From left to right: ddH2O, Py-20 (same sequence as lipo-20, but no lipid was coupled) in PBS buffer, lipo-20 in PBS and acetone mixture (v/v 50:50) and lipo-20 in PBS buffer. Samples were illuminated by a UV transilluminator (312 nm) and photographed by a digital camera. Panel (C) and (D) are 4% agarose gel analysis of amphiphilic DNA. From left to right: Py-20, lipo-5, lipo-10, lipo-20 and lipo-50. (C) Gel ran in 1×TBE buffer. (D) Gel ran in 1×TBE buffer containing 0.8% SDS.

FIG. 3 (A-E) is five panels. Panel (A) shows AFM topography image of the self-assembled micelle (lipo-20) deposit on a mica surface. The sample was pipetted onto a freshly cleaved mica surface and imaged by typing mode AFM in PBS buffer. Panels (B-E) show dynamic light scattering data of lipo-5 (B), lipo-10 (C), lipo-20 (D) and lipo-50 (E).

FIG. 4 (A-D) is four panels showing interactions between the DNA-micelles and CEM cells. Cells were treated with 200 nM DNA-micelles (top panels) and control (bottom panels) for 3 hours and subsequent examination by confocal microscopy. (A) and (C) Fluorescence images. (B) and (D) transmitted images. Flow cytometry data are shown below images. The bold numbers to the right of the histogram are the total mean fluorescence of the cell populations. The background fluorescence (untreated cells) was 4.76. (Scale bar: 50 μm.)

FIG. 5 (A-C) is three panels showing the localization and distribution of the DNA-micelles in CEM cells. CEM cells were treated with DNA-micelles (200 nM) and Tf-Alexa 633 (5 μg/ml) for 30 minutes. After washing, cells were imaged for the TAMRA fragment (A) and transferrin Alexa 633 (B); (C) overlay of a and b. (Scale bar: 20 μm.)

FIG. 6 is a graph showing fluorescence spectra of Nile red encapsulated in DNA micelles.

FIG. 7 is a graph showing Critical Micelle Concentration (CMC) determination of lipo-20 in 1×PBS buffer. Excitation was 350 nm.

FIG. 8 is two graphs (left and right) showing the results of thermodynamic study of lipo-20 in water (left) and in 1×PBS buffer (right). The numbers on the right indicate temperatures (° C.).

FIG. 9 is a graph showing interactions between DNA micelles and DSPC liposome. 2 μM lipo-20 was mixed with DSPC liposome (20 μM lipid) in PBS buffer, and the fluorescence spectra were measured at different time intervals.

FIG. 10 (A-C) shows engineered cell assembly through cell surface modification by aptamers. (A) is a schematic representation of cell-cell adhesion upon aptamer-protein binding. (B) shows illustrations of the schematic symbols used in (A). (C) shows the molecular structures of PEG linker and diacyllipid molecule.

FIG. 11 (A-E) shows aptamer directed multicellular assembly of 3-dimensional aggregate structures. Top panels: homotypic cell assembly. (A) Ramos cells spontaneously aggregate after treated with lipo-tdo5-TMR. (B) and (C) Control experiments show no assembly when Ramos cells were treated with lipo-lib-TMR (B) or CEM cells treated with lipo-tdo5-TMR (C). Bottom panels: heterotypic cell assembly. (D) 1:1 mixture of lipo-sgc8-TMR modified Ramos and CEM. (E) 1:10 mixture of lipo-sgc8-TMR modified Ramos and CEM. (F) Discrete cell clusters at higher magnification. (Scale bar: 100 μm)

FIG. 12 (A and B) shows Ramos cells aggregates disappear after treated with proteinase K. (A) Homotypic aggregates of Ramos after modified with lipo-tdo5-TMR; (B) The same assembled cells after incubation at 37° C. in the presence of Proteinase K. (Scale bar: 100 μm)

FIG. 13 (A-C) shows aptamer-mediated homotypic assembly of CEM cells. (A) CEM cells aggregate after treated with lipo-Sgc8-TMR; (B) CEM cells treated with lipo-lib-TMR; (C) Ramos cells treated with lipo-Sgc8-TMR. (Scale bar: 100 μm).

FIG. 14 (A and B) shows heterotypic assemblies between CEM and Ramos cells. (A) 1:1 mixture of lipo-Sgc8-TMR modified Ramos (red fluorescence) and CEM (nonfluorescent) cells after vigorously votex. Small cell clusters consist of both Ramos and CEM can be clearly observed. (B) 1:1 mixture of lipo-lib-TMR treated Ramos (red fluorescence) and CEM (nonfluorescent) cells. (Scale bar: 100 μm).

FIG. 15 (A and B) shows sequence specific heterotypic assemblies between CEM and Ramos. (A) 1:10 mixture of lipo-Tdo5-TMR modified CEM (red fluorescence) and Ramos (nonfluorescent) cells. (B) 1:10 mixture of lipo-lib-TMR modified CEM (red fluorescence) and Ramos (nonfluorescent) cells. (Scale bar: 100 μm).

FIG. 16 (A-D) shows sequence specific heterotypic assemblies between Jurkat and CEM/Ramos cells. (A) 1:10 mixture of lipo-Sgc8-TMR modified Jurkat (red fluorescence) and CEM (nonfluorescent) cells. (B) 1:10 mixture of lipo-lib-TMR modified Jurkat (red fluorescence) and CEM (nonfluorescent) cells. (C) 1:10 mixture of lipo-Tdo5-TMR modified Jurkat (red fluorescence) and Ramos (nonfluorescent) cells. (D) 1:10 mixture of lipo-lib-TMR modified Jurkat (red fluorescence) and Ramos (nonfluorescent) cells. (Scale bar: 100 μm).

FIG. 17 (A-D) shows sequence specific heterotypic assemblies between K562 and CEM/Ramos cells. (A) 1:10 mixture of lipo-Sgc8-TMR modified K562 (red fluorescence) and CEM (nonfluorescent) cells. (B) 1:10 mixture of lipo-lib-TMR modified K562 (red fluorescence) and CEM (nonfluorescent) cells. (C) 1:10 mixture of lipo-Tdo5-TMR modified K562 (red fluorescence) and Ramos (nonfluorescent) cells. (D) 1:10 mixture of lipo-lib-TMR modified K562 (red fluorescence) and Ramos (nonfluorescent) cells. (Scale bar: 100 μm).

FIG. 18 (A and B) shows assembling multiple types of cells. (A) Schematic diagram of assembly of multiple types of cells, lipo-tdo5-TMR modified K562 cells (red fluorescence) was first incubated with 5 eq lipo-sgc8-FAM modified Ramos (green fluorescence), then 5 eq of unmodified CEM cells was added. (B) Fluorescence microscopy image of the assemblies, multiple types of cells can be observed in the same aggregates. (Scale bar: 100 μm).

FIG. 19 (A and B) shows diacyllipid tail is required for a firm insertion. (A) Fluorescence microscopy image of CEM cells modified with monoacyllipid DNA (monolipid-lib-TMR). (B) Fluorescence microscopy image of CEM cells modified with diacyllipid DNA (dilipid-lib-TMR).

FIG. 20 (A-C) A) Schematic illustration of aptamer-micelle formation. All the related sequences are listed in Table 1 (and shown in FIG. 26). B). Flow cytometric assay to monitor the binding of free TDO5 (250 nM) and TDO5-lipid (250 nM) with Ramos cells (target cells) and HL60 (control cells) at 37° C. for 5 minutes. The blue and black curves represent the background binding of unselected DNA library and TDO5-micelle, respectively. The purple and red curves represent the binding of TDO5 and TDO5-micelle, respectively. C). Flow cytometric assay to monitor the binding of free aptamer (250 nM) and aptamer-lipid (250 nM) with target cells (K562) and control cell (CCRF-CEM) at 37° C. for 5 minutes. Neither FITC-KK nor FITC-KB aptamers bind (or bind only weakly) to the target cells. However, FITC-KK-micelle and FITC-KB-micelle show increased binding to the target cells. All probes (FITC-KK, FITC-KB, FITC-KK-micelle, FITC-KB-micelle) do not bind with control cells.

FIG. 21 (A and B) is a time course of displacement of FITC-TDO5 (A) or FITC-TDO5-micelle (B) bound onto the target cells by competition with an excess of non-labeled TDO5. Cells were incubated with binding buffer containing 250 nM FITC-labeled probes for 20 minutes at 4° C. Then 2.5 mM non-labeled TDO5 was added to the cells, and flow cytometric measurements were carried out at times as shown in the x-axis. The fluorescence intensity before the displacement was normalized to 100% binding. The fluorescence intensity of each data point was normalized to the binding percentage.

FIG. 22 (A-F) are six panels. (A) shows the design scheme of dye-doped micelles. Bright field and fluorescent images of Ramos cells after incubation with free CellTracker™ Green BODIPY for 2 hours (B) and 12 hours (C), or incubation with biotin-TDO5-micelle doped with CellTracker™ Green BODIPY for 2 hours (D). Image E is the enlarged fluorescent image after post-labeling the biotinylated TDO5 aptamer with QD705 streptavidin. The inset in image C is the enlarged individual cell image. The inset in image E is the fluorescent image of the dead cell. (F). Real-time monitoring of doped special dyes released from the core of the micelles and activated by intracellular enzymes.

FIG. 23 (A-D) are 4 panels, where (A) shows the enlarged fluorescence image, (B) shows the bright field image, and (C) shows the stack image after Z-depth scanning of Ramos cells after incubation with TMR-TDO5-micelle in complete cell medium at 37° C. for 2 hours. The cross mark in image C indicates that the brightest fluorescence signal comes from inside the cell. (D) Co-localization of TMR-TDO5-micelle (red) and AF633-transferrin (blue) in endosomes.

FIG. 24 (A-D) shows flow cytometric assay to monitor the binding of 250 nM TDO5-micelle or library-micelle (based on lipid unit concentration) with cell mixture made by spiking 1 million Ramos (target cells, A, C & D) or HL60 (control cells, B) in 50 uL male whole blood. The incubation time varied from 5 minutes (A & B) to 1 hour (C) and 2 hours (D). The green and red curves represent the binding of unselected DNA library-micelle and TDO5-micelle, respectively.

FIG. 25 (A-D) shows simplified flow channel response to cell staining assay. (A) Stepwise immobilization scheme of the flow channel. Representative images of the bright field and fluorescent images of control cells (CCRF-CEM) and target cells (Ramos) captured on the flow channel surface incubated with FITC-TDO5-micelle (B), or FITC-library-micelle (C) or free FITC-TDO5 (D) spiked in human whole blood sample under continuous flow at 300 mL/s at 37° C. for 5 minutes. All the scale bars are 100 μm.

FIG. 26 is a Table (Table 1) that shows the oligonucleotides employed in the present experiments.

FIG. 27 (A-C) is three panels that show the amphiphilic unit self-assembled into a spherical micelle structure, as demonstrated in the TEM image (FIG. 27). The aptamer used in this case is called TDO5, which was selected specific to Ramos cells (a B-cell lymphoma cell line) (Tang, 2007). As shown in FIG. 27B, the TDO5-micelle has an average diameter of 68±13 nm, which is consistent with the hydrodynamic diameter measured by Dynamic Light Scattering of 67.22 nm (FIG. 27C).

FIG. 28 (A and B) shows the dissociation constants of the aptamer-micelles.

FIG. 29 (A and B) shows rapid identification with high sensitivity. TDO5-micelle demonstrated extremely rapid recognition of the target cells. A, after an incubation of only 30 sec at 37° C., a 45-fold enhancement in fluorescence intensity when binding with target cells was observed for TDO5-micelle.

FIG. 30 (A-C) shows trace cell detection in whole blood sample.

FIG. 31 (A-C) shows aptamer-micelle targeting in a flow channel under a continuous flow. A representative result after micelle-buffer incubation with FITC-TDO5-micelle is shown in FIG. 31.

FIG. 32 (A and B) shows that the aptamer-micelle was found to have low CMC and low cytotoxicity to the cells.

FIG. 33 (A and B) are two panels that show results of flow cytometry experiments.

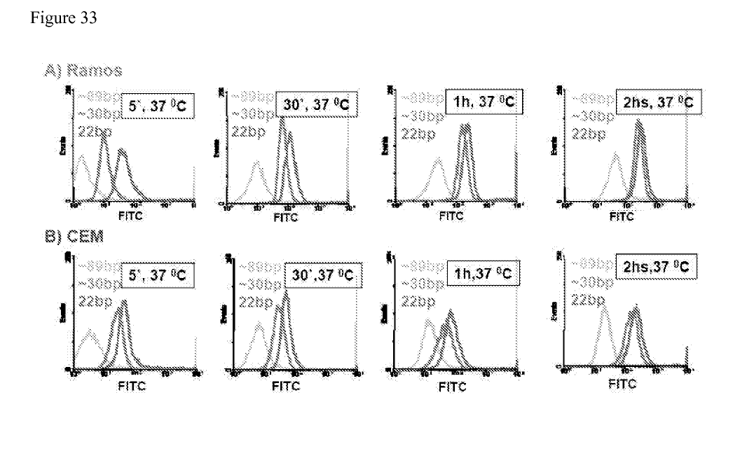

FIG. 34 (A and B) are two panels showing experiments with micelles where PEG-lipid was replaced with Macugen-lipid.

FIG. 35 (A-C) are three panels, where panel (A) is a schematic, and panel (B) and (C) are graphs.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “agent” is meant to refer to a drug, a compound, a polypeptide, a polynucleotide, or fragment, or analog thereof, a small molecule, or any other biologically active molecule.

The term “amphiphilic” as used herein refers to having both hydrophilic and hydrophobic properties.

The term “antisense,” as used herein, refers to any composition containing a nucleic acid sequence which is complementary to a specific nucleic acid sequence. The term “antisense strand” is used in reference to a nucleic acid strand that is complementary to the “sense” strand. Antisense molecules may be produced by any method including synthesis or transcription. Once introduced into a cell, the complementary nucleotides combine with natural sequences produced by the cell to form duplexes and to block either transcription or translation.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783 21789, Delihas et al., 1997, Nature, 15, 751 753, Stein et al., 1997, Antisense N. A. Drug Dev., 7, 151, Crooke, 2000, Methods Enzymol., 313, 3 45; Crooke, 1998, Biotech. Genet. Eng. Rev., 15, 121 157, Crooke, 1997, Ad. Pharmacol, 40, 1 49.

The terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “DNAzyme” as used herein is meant to refer to a catalytic DNA molecule. In certain embodiments, DNAzymes are capable of specific catalysis of target mRNA

The term “homogenous” as used herein is used to refer to a population of micelles that is suitably of uniform structure or composition. In preferred embodiments, the homogenous population shares suitably uniform size. In other preferred embodiments, the homogenous population shares suitably uniform weight.

The term “loaded” is meant to refer to a micelle that has an agent, for example a therapeutic agent, or a drug, situated within the center or core, the hydrophobic core, of the micelle.

The term “micelle”, as used herein, refers to any water soluble aggregate which is spontaneously and reversibly formed from amphiphilic compounds or ions.

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

The term “pharmaceutically-acceptable carrier” as used herein means one or more compatible solid or liquid filler, diluents or encapsulating substances that are suitable for administration into a human.

The term “siRNA” is meant to refer to small interfering RNA; a siRNA is a double stranded RNA that “corresponds” to or matches a reference or target gene sequence. This matching need not be perfect so long as each strand of the siRNA is capable of binding to at least a portion of the target sequence. SiRNA can be used to inhibit gene expression, see for example Bass, 2001, Nature, 411, 428 429; Elbashir et al., 2001, Nature, 411, 494 498; and Zamore et al., Cell 101:25-33 (2000).

The term “small molecule” inhibitor is meant a molecule of less than about 3,000 daltons having tyrosine kinase antagonist activity.

The term “subject” is intended to include vertebrates, preferably a mammal. Mammals include, but are not limited to, humans.

The term “target cell” is meant to refer to an individual cell or cell which is desired to be, or has been, a recipient of micelles. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism. As used herein, a “target cell” is generally distinguished from a “host cell” in that a target cell is one which is found in a tissue, organ, and/or multicellular organism, while as host cell is one which generally grows in suspension or as a layer on a surface of a culture container.

### DETAILED DESCRIPTION OF THE INVENTION

The present invention is based on the design and construction of a series of well-defined self-assembled oligonucleotide micelles by conjugating hydrophilic DNA with hydrophobic lipid tails. The invention describes that, in aqueous solutions, the micelles spontaneously self-assemble into monodispersed micelle structures with a lipid core and a DNA corona. The invention describes micelles with superior thermodynamic stability. The size of the micelles described by the instant invention can be fine tuned by changing the length of the DNA sequence and can disassemble themselves for insertion into a model membrane. Taken together, the micelles described herein by the instant invention have considerable cell permeability, and are suitable for a variety of applications in nanobiotechnology, cell biology and gene delivery systems

### I. Compositions

The present invention is directed to homogenous populations of micelles. The population of micelles comprise oligonucleotides and hydrophobic lipid elements that, in certain examples, are covalently linked each other.

A micelle is spontaneously formed by self-assembly of molecules having both hydrophilic and hydrophobic moieties at a specific ratio in an aqueous environment to maximize thermodynamic stability. In further embodiments, the inside of the micelles is hydrophobic and thus can easily entrap water-insoluble drugs, and the surface of the micelles is hydrophilic and thus the micelle system facilitates solubilization of the water-insoluble drugs, drug delivery carrier and so on. Micelles having the hydrophobic core and the hydrophilic shell are stabilized in an aqueous environment by hydrophobic interaction, or stabilization of micelles can be achieved by ionic interaction between polyelectrolytes having opposite charges.

It is a novel finding of the instant invention that the population of micelles is a homogenous population.

The population of micelles as described is comprised of oligonucleotides and hydrophobic lipid elements. Hydrophobic lipid elements can be any one of linear, branched chain or cyclic. Preferably, the hydrophobic element is between 8, 9, 10, 11, 12, 13, 14, 15, 16, 17, 18, 19, 20, 21, 22, 23, 24, and 25 units in length. In exemplary embodiments, the hydrophobic element is 18 units in length. In Preferably, the chain elements of said hydrophobic element are carbon atoms.

In preferred embodiments of the invention, the population of micelles is homogenous in diameter, or size. The diameter may also be referred to as “hydrodynamic diameter.” Hydrodynamic diameter of a micelle indicates that the micelle has the same hydrodynamic properties (e.g., diffusion coefficient) as a sphere of the same diameter. For example, a micelle having a width of 5 nm and a length of 9 nm might have a hydrodynamic diameter of 7 nm.

The hydrodynamic diameter of the micelles of the present invention can be from about 1 nm to about 100 nm, preferably from about 3 nm to about 50 nm.

The hydrodynamic diameter of micelles also means that considerably all the micelles have about the same hydrodynamic diameter (i.e., or range of hydrodynamic diameters as described above); for example more than 50% of the micelles have a hydrodynamic diameter that falls within the range as described above; preferably more than 60%, 70% or 80% of the micelles have a hydrodynamic diameter that falls within the range as described above; even more preferably about 90%, about 95%, or about 99% of the micelles have a hydrodynamic diameter that falls within the range as described above; most preferably 100% of the micelles have a hydrodynamic diameter that falls within the range as described above.

In other preferred embodiment, the population is homogenous in weight. For example, the weight can range from about 500 to 75,000, preferably 1,000 to 50,000 Da in weight.

In certain embodiments, the oligonucleotides comprise single stranded DNA. The single stranded DNA can be between 2-100 nucleotide bases in length, for example, 5 nucleotide bases in length, 10 nucleotide bases in length, 15 nucleotide bases in length, 20 nucleotide bases in length, 25 nucleotide bases in length, 30 nucleotide bases in length, 350 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

In preferred examples, the oligonucleotide is selected from any one of, but not limited to, an antisense oligonucleotide, a decoy oligonucleotide, a siRNA, a DNAzyme, a ribozyme, and an aptamer. The oligonucleotides may further comprise naturally occurring or modified nucleosides such as DNA, RNA, locked nucleic acids (LNA's), 2′O-methyl RNA (2′Ome), 2′O-methoxyethyl RNA (2′MOE) in their phosphate or phosphothioate forms or Morpholinos or peptide nucleic acids (PNA's).

For example, non-limiting examples of the oligonucleotide can be an oligonucleotide that decreases or down-regulates expression of, or is originated from, any gene having possibility of using in delivering an oligonucleotide, for example an siRNA.

In other preferred embodiments, the oligonucleotide decreases expression of a target gene.

The oligonucleotides and hydrophobic elements are linked, preferably by a covalent bond. The covalent bond may be a non-cleavable linkage. In other examples, the covalent bond may be a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

Preferably, in certain examples, the population of micelles further comprises reporter molecules. The reporter molecules, for example, are fluorescent. Examples include, but are not limited to pyrene.

Certain drugs are poorly soluble in water, or other common vehicles used for parenteral administration. Further, conventional formulations have resulted in unwanted clinical side effects.

Generally, micelles can solubilize otherwise insoluble organic material by incorporating the organic material within their hydrophobic interior. Particularly useful in the compositions described are hydrophobic agents, in particular hydrophobic drugs.

Thus, in certain embodiments the invention features a population of micelles further comprising an agent. The agent is loaded into the hydrophobic core of the micelle. As described above, a pharmaceutical formulation can be formed from the composition by adding an aqueous solution such as water, saline or any other suitable other infusion fluid. When an aqueous solution is added, hydrophobic tails entrap the agent within a micelle, thereby solubilizing the agent in the resultant pharmaceutical formulation.

The compositions are useful for delivering agents that are insoluble in water or other aqueous solvents or that, when administered in conventional formulation, result in unwanted side effects. When the drug is in the form of a micelle, the effectiveness of delivery of the drug to the site of action may be affected by the size of the aggregate, as the micelle size might affect diffusion, transport across cell membranes, and interactions with enzymes, transport proteins and lipids. Thus, a homogenous population, as described herein, provides more controlled delivery and/or pharmokinetics.

The agent may be any therapeutic agent. The agent may be any one of, but not limited to, a drug, a toxin, a gene, a small molecule and an oligonucleotide. The oligonucleotide may be, for example, an antisense oligonucleotide, a decoy oligonucleotide, a siRNA, a DNAzyme, a ribozyme, and an aptamer

Particularly useful in the compositions described are hydrophobic drugs. Exemplary drugs may be chemotherapeutic agents, for example, but not limited to, paclitaxel, cisplatin, or doxorubicin.

In other embodiments, the agent may be an imaging agent.

The solvent in the composition for delivering the composition in vivo may preferably be pharmaceutically acceptable, water miscible, nonaqueous solvent. In the context of this invention, these solvents should be taken to include solvents that are generally acceptable for pharmaceutical use, substantially water-miscible, and substantially non-aqueous. Preferably, these solvents do not cause phthalate plasticizes to leach when the solvents are used with medical equipment whose tubing contains phthalate plasticizers. Preferred examples of the pharmaceutically-acceptable, water-miscible, non-aqueous solvents that may be used in this invention include, but are not limited to, N-methylpyrrolidone (NMP); propylene glycol; polyethylene glycol (e.g. PEG300, PEG400, etc.); ethyl acetate; dimethyl sulfoxide; dimethyl acetamide; benzyl alcohol; 2-pyrrolidone; benzyl benzoate; C.sub.2-6 alkanols; 2-ethoxyethanol; alkyl esters such as 2-ethoxyethyl acetate, methyl acetate, ethyl acetate, ethylene glycol diethyl ether, or ethylene glycol dimethyl ether; (s)-(−)-ethyl lactate; acetone; glycerol; alkyl ketones such as methylethyl ketone or dimethyl sulfone; tetrahydrofuran; cyclic alkyl amides such as caprolactam; decylmethylsulfoxide; oleic acid; aromatic amines such as N,N-diethyl-m-toluamide; or 1-dodecylazacycloheptan-2-one.

Most preferred examples of pharmaceutically-acceptable, water-miscible, non-aqueous solvents include alcohols such as ethanol, propylene glycol and benzyl alcohol; polyalcohols such as polyethylene glycol (PEG 300, PEG 400, etc.); and amides such as 2-pyrrolidone, N-methyl-pyrrolidone and N,N-dimethyl acetamide. Additionally, triacetin may also be used as a pharmaceutically-acceptable, water-miscible, non-aqueous solvent, as well as functioning as a solubilizer in certain circumstances.

The pharmaceutical composition as described herein may optionally further include an excipient added to the composition in an amount sufficient to enhance the stability of the composition, maintain the product in solution, or prevent side effects associated with the administration of the inventive composition. Examples of excipients include but are not limited to, cyclodextrin such as .alpha.-, .beta.-, and .gamma.-cyclodextrin and modified, amorphous cyclodextrin such as hydroxy-substituted .alpha.-, .beta.-, and .gamma.-cyclodextrin. Cyclodextrins such as ENCAPSIN from Janssen Pharmaceuticals may be used for this purpose.

The composition may be incorporated into a pharmaceutical carrier suitable for oral administration. In a preferred embodiment, polyethylene glycols, such as PEG 300 and 400, may be used as the solvent for their capability of solubilizing paclitaxel and forming semi-solid to solid compositions. In this embodiment, the concentration of polyethylene glycol may preferably be less than about 40% w/w in the finally formed composition. The composition may be filled into a soft or hard gelatin capsule, or another suitable oral dosage form with protective or sustained release coatings and orally administered into a host in need thereof, such as a cancer patient.

The types of protective or sustained release coating that may be used include, but are not limited to, ethylcellulose, hydroxypropylmethylcellulose, hydroxypropylcellulose, hydroxyethylcellulose, and esters of methacrylic and ethacrylic acid (Eudragit RL, RS, and NE polymer products, Rohm Pharma, Darmstadt, Germany). The enteric protective materials or coatings may be, for example, cellulose acetate pthalate, hydroxypropylmethylcellulose, ethylvinylacetate pthalate, polyvinylacetate pthalate and esters of methacrylic and ethacrylic acid (Eudragit S, Eudragit L and Eudragit E30D, Rohm Pharma, Darmstadt, Ger.).

Alternatively, the composition may also be diluted into an aqueous solution to form a pharmaceutical formulation by adding saline or other infusion fluid for parenteral administration or intravenous injection. The pharmaceutical formulation will be described in details below.

### II. Methods

In one aspect, the invention features methods of preparing a homogenous population of micelles comprising preparing oligonucleotides, preparing hydrophobic lipid elements; and mixing oligonucleotides and hydrophobic lipid elements in an aqueous medium, thereby forming a homogenous population of micelles.

In further embodiments, the method further comprises mixing reporter molecules. As described, the reporter molecules may be fluorescent (e.g. pyrene).

The aqueous medium can be any aqueous medium that is suitable for the formation of micelles, for example, but not only limited to, water or saline.

In one example of a method of preparing such a micelle, the DNA-lipid conjugate is mixed with a phosphate buffer and the micelle is spontaneously formed. In certain embodiments, to stabilize the micelle, the mixture is laid aside at room temperature for a period of time, for example 30 minutes.

In certain examples, the invention features methods of delivering an oligonucleotide to a target cell comprising contacting the target cell with a population of micelles comprising oligonucleotides and hydrophobic lipid elements; and incubating the target cell with the population of micelles; thereby delivering an oligonucleotide to the target cell.

In other examples the invention features methods of delivering an oligonucleotide to a subject comprising administering to the subject a population of micelles comprising oligonucleotides and hydrophobic lipid elements, thereby delivering an oligonucleotide to a subject.

In preferred embodiments, the oligonucleotide is selected from, but not only limited to, an antisense oligonucleotide, a decoy oligonucleotide, a siRNA, a DNAzyme, a ribozyme, and an aptamer. In further preferred embodiments of the invention, the oligonucleotide is a therapeutic oligonucleotide.

Gene therapy is a new era of biomedical research aimed at introducing therapeutic genes into somatic cells of patients (reviewed by Boulikas, 1998a; Martin and Boulikas, 1998). Two major obstacles prohibit successful application of somatic gene transfer: (1) the small percentage of transduced cells and (2) the loss of the transcription signal of the therapeutic gene after about 3-7 days from injection in vivo.

The first problem arises (i) from inability of delivery vehicles carrying the gene to reach the target cell surface (the vast majority of liposome-DNA complexes are eliminated from blood circulation rapidly); (ii) from difficulty to penetrate the cell membrane and (iii) to release the DNA from endosomes after internalization by cells; (iv) from inefficient import into nuclei. Others have used stealth liposomes (Martin and Boulikas, 1998a), which persist in circulation for days and concentrate in tumors. However, classical stealth liposomes are not taken up by cancer cells. The invention described herein described novel and effective methods of delivery of nucleic acids.

The second problem results from the loss of the plasmids in the nucleus by nuclease degradation and failure to replicate autonomously leading to their dilution during cell proliferation among progeny cells or by inactivation of the foreign DNA after integration into the chromosomes of the host cell. However, the use of human sequences able to sustain extrachromosomal replication of plasmids for prolonged periods (see U.S. Patent on “Cloning method for trapping human origins of replication” by Teni Boulikas U.S. Pat. No. 5,894,060) will overcome this limitation.

In certain aspects the invention features methods of delivering nucleic acids to a target cell comprising contacting the target cell with a population of micelles comprising nucleic acids and hydrophobic lipid elements and incubating the target cell with the population of micelles thereby delivering nucleic acids to the target cell.

In another aspect the invention features methods of delivering nucleic acids to a subject comprising administering to the subject a population of micelles comprising nucleic acids and hydrophobic lipid elements and thereby delivering nucleic acids to the subject.

In certain aspect, the invention features the therapy of subject, e.g., mammals such as mice, rats, simians, and human patients, with a disease or disorder. In particular, the invention features methods for treating a disease or disorder in a subject comprising administering to the subject a population of micelles comprising oligonucleotides and hydrophobic lipid elements, thereby treating the disease or disorder in the subject.

The method is useful, for example, in treating cancers, for example human cancers including, but not limited to breast, prostate, colon, non-small lung, pancreatic, testicular, ovarian, cervical carcinomas, head and neck squamous cell carcinomas.

Any of the methods as described herein can be practiced in vitro, ex vivo or in vitro.

### Patient Monitoring

The disease state or treatment of a patient undergoing treatment with the compositions of the invention can be monitored. In one embodiment, the delivery of oligonucleotide to the subject can be monitored by detection of the reporter (for example, but not limited to, pyrene). For example, delivery of the oligonucleotide or the nucleic acid to a target cell, for example a tumor cell, can be monitored.

Such monitoring may be useful, for example, in assessing the efficacy of a particular oligonucleotide or nucleic acid therapy. Accordingly, the oligonucleotide can be any one of, but not limited to, an antisense oligonucleotide, a decoy oligonucleotide, a siRNA, a DNAzyme, a ribozyme, or an aptamer, as long as the target is therapeutically relevant or of interest, or provides a benefit to the subject (e.g. alleviates a disease or disorder).

### III. Formulation and Administration

The pharmaceutical formulation can be used for delivering populations of micelles as described herein in vivo. The pharmaceutical formulations according to the invention may be administered in any medically suitable manner, preferably parenterally or orally, more preferably parenterally, and still more preferably intravenously.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous isotonic sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders of the kind previously described.

When using a lyophilized drug product, clinicians typically reconstitute the freeze-dried preparation in physiologically acceptable solutions. It is desirable to be able to store the reconstituted solution either at room temperature or under refrigeration. Freeze-dried preparations of the micelles described herein are rehydratable with water or an aqueous dextrose solution suitable for intravenous administration, with the micelle hydrodynamic diameter distribution remaining unchanged. Such reconstituted micelle solutions can be stored at room temperature or refrigerated temperatures with no change in the micelle hydrodynamic diameter.

### IV. Kits

In yet another aspect, the invention provides kits. Preferably, the kits comprise oligonucleotides and hydrophobic lipid elements, as described herein. The kits also contain instructions for use.

For example, in certain embodiments, the kit may include instructions for use in preparing a homogenous population of micelles. In other embodiments, the kit may include instructions for use in delivering an oligonucleotide to a cell. In other embodiments, the kit may include instructions for use in delivering an oligonulcoetide to a subject. In other embodiments, the kit may include instructions for use in delivering nucleic acids to a cell. In other embodiments, the kit may include instructions for use in delivering nucleic acids to a subject. In other embodiments, the kit may include instructions for use in treating a disease or disorder in a subject. In other embodiments, the kit comprises a sterile container which contains the oligonucleotides; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the oligonucleotides described herein and their use in diagnosing a neoplasia. In other embodiments, the instructions include at least one of the following: description of the use of a reporter, for example pyrene; precautions; warnings; indications; clinical or research studies; and/or references. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. It will be apparent to those skilled in the art that various modifications and variations can be made in the compositions, kits, and methods of the present invention without departing from the spirit or scope of the invention. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

The construction of complex supramolecular structures from simple amphiphilic molecules via molecular self-assembly has drawn much attention in recent years. (1-4) Amphiphilic peptides, (1) nucleic acids, (2) lipids (3) and polymers (4) have been shown to generate ordered supramolecular structures such as monolayers, micelles, vesicles, bilayers and nanotubes. For biomedical applications, micelle structures are of particular interest because of their small size, good biocompatibility, high stability both in vitro and in vivo, and the ability to carry poorly soluble pharmaceuticals.

While a variety of micelle systems have been demonstrated, (1-4) the design and construction of such self-assembled micelle systems for nanobiotechnological applications requires precise molecular engineering of the corresponding building blocks, since all the information required for the assembly must be encoded in their molecular architecture.

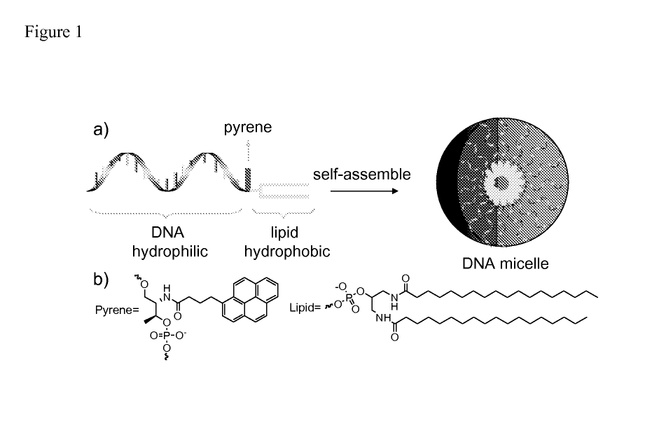

In one embodiment, the invention described herein features micelles comprising oligonucleotides and hydrophobic elements. The micelles can also include a fluorescent reporter. FIG. 1 is a schematic of an exemplary micelle of the invention as described herein. As shown in FIG. 1, the micelle system includes oligonucleotides, for example DNA, which is highly hydrophilic. In certain embodiments, DNA was chosen as a micelle-forming material because of its well-defined structures as well as its versatile chemical and biological functions. Another component of the micelle shown in FIG. 1 is a pyrene molecule. Here, a pyrene unit acts as fluorescence reporter because pyrene has unique fluorescence characteristics which have been widely used to probe the aggregation behavior of various systems. A highly hydrophobic third component is composed of hydrocarbon tails, for example two C18 hydrocarbon tails, as shown in FIG. 1. In water, the hydrophobic effect is the driving force for micelle formation. In one embodiment, the DNA-lipid molecules are able to self-assemble as a result of hydrophobic effect. In another embodiment, both the size and the function of the micelles can be readily adjusted by fine tuning the DNA corona, which provides unique control in the construction of functional nanostructures. The DNA-Micelle system can be easily prepared in situ, and DNA-micelles have very low critical micelle concentration (for example, below 10 nM) and maintain their integrity even at temperatures of 95° C.

### Example 1

### Oligonucleotide Micelles—Preparation

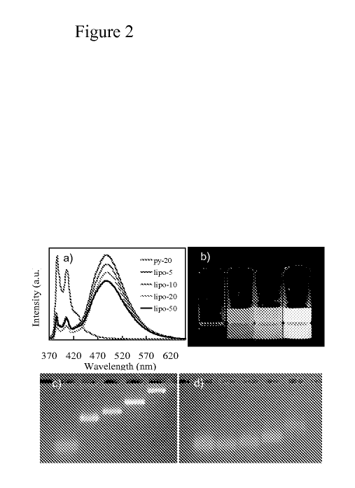

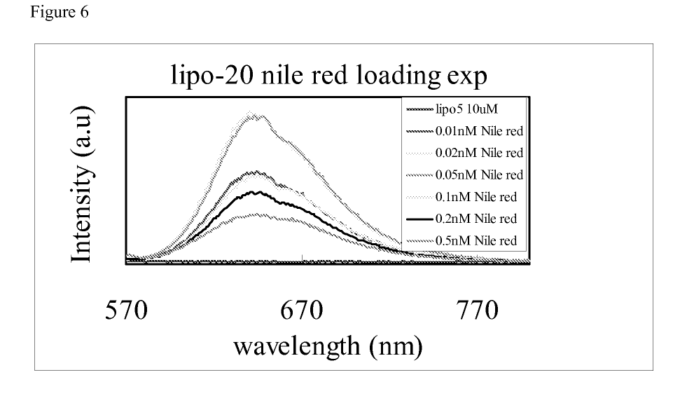

The amphiphiles were prepared in high yield through solid phase synthesis on controlled pore glass beads (CPG) (see Methods). Four amphiphiles with different DNA lengths (random sequences, lipo-n, where n denotes the length of oligonucleotides) were prepared and used for the self-assembly. In aqueous solutions, these DNA amphiphiles spontaneously self-assembled into three-dimensional spherical micelles with a DNA corona and a lipid core. The self-assembly characteristics were then investigated by first employing fluorescence techniques. In the aggregation state, the pyrene units, which were designed to be close to the lipid tails, are spatially proximal to each other and give excimer-type fluorescence of pyrene. (Conlon et al. 2008) All four DNA-micelles used in this study revealed a broad emission of pyrene excimer at 490 nm with an excitation at 350 nm in aqueous solution, as shown by the fluorescent spectra in FIG. 2a. This result shows the strong π-π excitonic interactions of the pyrene chromophores in the micelle systems, which indicates the well-organized state of the assembled micelles. In addition, when a DNA without lipid (py-20; same sequence as lipo-20, but without lipid coupling) was tested, no excimer fluorescence was observed (FIG. 2a, b). The hydrophobic microenvironment of DNA-micelles was also investigated with Nile red, a polarity-sensitive fluorescent dye which acts as a hydrophobic probe. Its fluorescent maxima vary depending on the relative hydrophobicity of the surrounding environment. Thus, encapsulated Nile red fluorescence at 637 nm and excitation at 550 nm suggests a relatively hydrophobic environment7 (FIG. 6). Amphiphilic assemblies normally form in response to solvent compositional change. FIG. 2b shows images of micelles in different solvent systems. As expected, the micelle structures are disrupted when acetone (a good solvent for lipid) was added as a cosolvent. The above results suggest the formation of aggregates when the DNA amphiphiles were dispersed in water. Importantly, the hybridization of the complementary DNA by formation of Watson-Crick base pairs does not seem to affect the aggregation, which suggests that it is possible to further manipulate these assemblies. Taken together, the fluorescence outcome matches a key objective of the design strategy because it proves the desired aggregation assemblies.

### Example 2

### Oligonucleotide Micelles—Self Assembly

Unlike previously reported DNA-polymer conjugates which have a range of molecular weights and sizes, (Jeong et al., 2001; Li et al., 2004; Safak et al. 2007) the micelles described herein have a precise molecular architecture. Therefore, further experiments were carried out to test whether the DNA amphiphiles could self-assemble into well-defined, homogeneous micelles. Agarose gel electrophoresis experiments were conducted. Gel electrophoresis is a powerful technique in biology and is the standard method used to separate, identify and purify nucleic acid with different sizes. It was hypothesized that micelle aggregation in the gel matrix would result in slow moving bands with green fluorescence (pyrene excimer). On the other hand, if aggregations were to be disrupted, it was further hypothesized that only fast moving bands with violet fluorescence (monomeric pyrene) would be observed. In addition, a sharp or condensed band would be consistent with uniform size and unique conformation. Since the mobility of the DNA amphiphiles solely depended on the length of the DNA, shorter DNA assemblies would generally be expected to migrate faster in the gel. As shown in FIG. 2c, in TBE buffer, each DNA assembly migrated as a single, sharp band with expected mobility, suggesting that the micelle aggregations were stably formed. It should be noted that the self-assembled structures exhibited single sharp bands for all of the assemblies, indicating that all the micelles are uniform in aggregation number. Nonetheless, attempts to estimate the aggregation number by a reported method (Tummino et al., 1993) yielded inconclusive results, which most likely resulted from interference of the highly charged DNA. To further prove that the slow mobility was a result of aggregation, 0.8% (w/v) Sodium dodecyl sulfate (SDS) was added to the gel. At this concentration, SDS can disrupt micelles, resulting in a loss of hydrophobic interactions. (Smiddy et al., 2006) The results showed that, under UV illumination, all the DNA migrated as faster single violet (monomeric pyrene) bands (FIG. 2d).

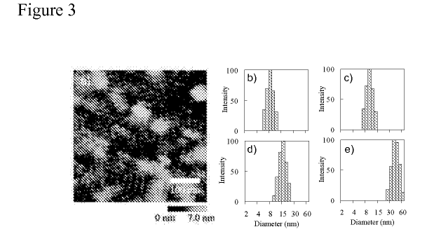

Next, to provide direct evidence of amphiphilic DNA self-assembly into micelles, the sample was imaged using tapping mode atomic force microscopy (AFM). AFM images revealed a dense layer of highly uniform spherical particles (FIG. 3a). The morphological characteristics of the DNA micelles under AFM were quite different from those of the synthetic diblock polymer micelles. The height measured by AFM for the micelles was about 1.2 nm, a number about half that of a double-strand DNA measured in liquid. (Liu et al., 2006) However, strong electrostatic interactions with the substrate and the vertical forces in tapping mode AFM commonly deform soft materials, leading to compressed morphologies. (Liang et al., 2004) Therefore, an alternate method was used, dynamic light scattering (DLS), which provides a fast, direct measurement of the physical sizes, as well as aggregation data for the dissolved DNA micelles (FIG. 3). The hydrodynamic diameters in PBS buffer, as measured by DLS, for lipo-5, lipo-10, lipo-20 and lipo-50, were 7.8 nm, 9.5 nm, 14.6 nm and 36.4 nm, respectively. These values agree well with the sizes of micelle aggregations, but are far less than those in previously reported DNA vesicles. (Dentinger et al. 2006) Despite the unknown conformation and persistence data for ssDNA, micelle size that is linearly proportional to DNA length suggests a predictable relationship.

### Example 3

### Oligonucleotide Micelles—Stability

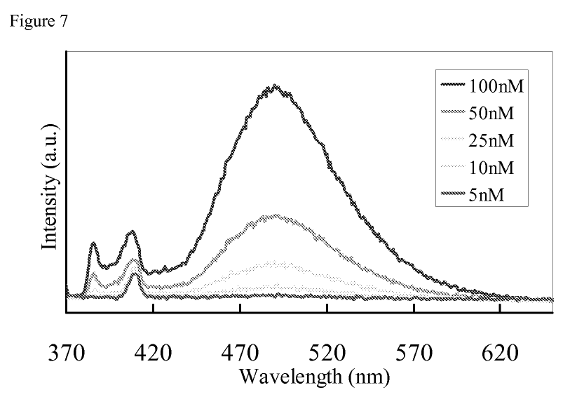

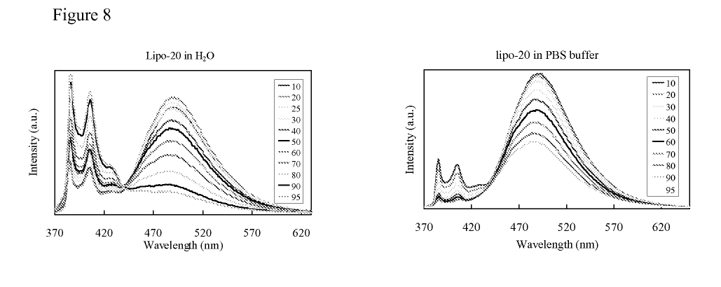

The DNA-micelle's stability was then investigated. It was found that all DNA-micelle solutions had very low critical micelle concentrations, (below 10 nM; FIG. 7). Because of the limited fluorescence of pyrene, it was noted that 10 nM should be considered the upper limit of CMC, rather than the actual values. Nevertheless, these extremely low CMCs indicate excellent stability compared to polymetric micelle systems. (Lukyanov et al., 2002) The thermodynamic stability of DNA-micelles was also investigated. In the presence of counterions (1×PBS buffer, 137 mM Na+, 2.7 mM K+), DNA-micelles maintained their integrity (excimer-type fluorescence peak), even at 95° C. When the temperature study was conducted in pure water, however, the excimer-type fluorescence vanished as temperature increased, while the monomeric fluorescence increased its intensity, showing a ratiometric response (FIG. 8). These data indicate that counterions can greatly stabilize the DNA-micelles, most likely by minimizing the anionic charge repulsion between DNA chains. It is possible that the stability of the DNA-micelles partially arises from the design itself, where the covalent linkage of two dramatically distinct segments prevents the dissociation and breakup of hydrophobic interactions. Finally, the micelles are stable for at least six months without formation of any precipitation when stored at 4° C.

### Example 4

### Oligonucleotide Micelles—Membrane Interaction

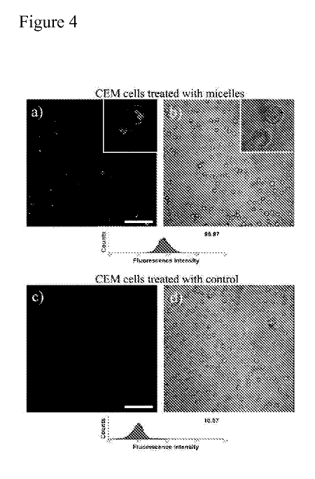

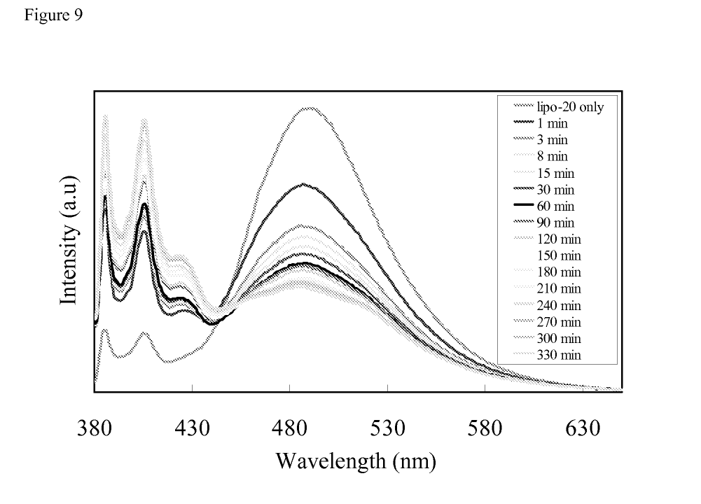

DNA modified with lipophilic moieties, such as cholesterol (Letsinger et al, 1989) or dendrimer (Skobridis et al., 2005), have shown enhanced cellular uptake via a receptor-mediated mechanism or by increased membrane permeability. In addition, nanometer-sized assemblies have functional and structural properties that are not available from either isolated molecules or bulk materials. (Niemeyer et al. 2001) Since efficient transport of nucleic acids across cell membranes is of great importance for antisense therapy, experiments were next designed to understand the interactions between the DNA-micelles and cell membranes. To accomplish this, first investigations were carried out to examine the interaction using a model membrane. Briefly, 2 μM lipo-20 micelles were mixed with small unilamellar vesicles (SUVs, 20 μM DSPC) in PBS buffer, and the pyrene fluorescence was monitored at different time intervals. A ratiometric fluorescence change (excimer fluorescence decreases while monomer fluorescence increases) was observed (FIG. 9), indicating that slow fusion occurred between micelles and vesicles. Because this result suggests that the micelles dissociate themselves and insert into the model membrane spontaneously, next, the affinity between the DNA-micelles and a plasma membrane was investigated. A 3′-TAMRA-labeled DNA-micelle (lipo-20-TMR, 20 nt; same sequence as lipo-20) was incubated with cultured human T lymphoblast cell line from acute lymphoblastic leukemia (CCRF-CEM cells) for three hours. Confocal microscopy and flow cytometry were used to image and quantify the fluorescent profiles. The same DNA sequence, but lacking the lipid moiety, was used as a control. CEM cells treated with DNA-micelles were highly fluorescent when compared to those treated with the controls (FIG. 4). To quantify the fluorescence, flow cytometry was used for a statistical analysis. The data revealed that CEM cells treated with DNA-micelles were 8.1 times more fluorescent than the population treated with control. The efficiency may be further optimized by varying many parameters of the internalization experiments. The flow cytometry data were found to be in agreement with confocal imaging (FIG. 4).

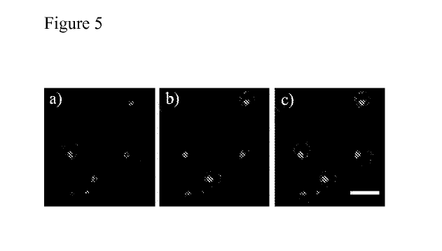

A higher magnification image reveals the cellular distribution of the DNA (FIG. 4a, inset). The fluorescence DNA was found to be uniformly distributed on the plasma membrane and also accumulated inside the cells. Next, the cellular locations of the internalized DNA was examined. Co-localization of the DNA-micelles via a better-characterized endocytic pathway, namely transferrin receptor (TfR)-mediated endocytosis, (Yang et al., 2006) was examined by confocal microscopy. As shown in FIG. 5, the cell fluorescence after treatment with DNA-micelles was found to co-localize with Tf-containing endosomes, suggesting that the DNA accumulates in endosomes. A confocal time course study revealed that the fluorescence first enriches on the cell membrane and is then internalized, most likely by endocytosis. It may be thought that the finding of DNA/TfR co-localization within the cell vesicular compartment, plus the fusion between DNA-micelles and vesicles, represents an important step in understanding the components of the mechanism for amphiphilic DNA uptake. It is possible that the micelles first disassemble themselves and then insert into the cell membrane or that the micelles cross the membrane as a whole structure. Either way, the fact that fluorescence accumulates in endosomes suggests an endocytic pathway for nucleic acid delivery into intracellular regions.

The experiments and results described herein feature the design and construction of highly stable, well-defined oligonucleotide micelles; these micelles have a hydrophobic lipid core and a hydrophilic DNA corona. The results presented herein showed that the DNA-micelles interact with cell membrane and can be internalized by endocytosis. This approach can be applied to any type of functional oligonucleotide (aptamer, DNAzyme) (Lu et al., 2006), making it attractive for the assembly of functionally controlled structures. Accordingly, the monodispersed size, high stability, and cell permeability of this class of micelles will find applications in nanobiotechnology, cell biology, and drug delivery.

### Example 5

### Aptamer Mediated Cell Assembly

Rapid and selectively assembling multiple types of cells into three-dimensional (3-D) architecture is integral to tissue engineering and tissue regeneration. It is also highly useful for therapeutical strategies and cell-cell communication studies. The present experiments report a versatile strategy for controlling cell-cell adhesion via a facile DNA modification of live cell surface that enables the rapid and controlled formation of specific 3-D interactions for a range of cell types. The present experiments show that aptamers anchored on the cell surface can act as artificial cell adhesion molecules (CAMs) that recognize their target cells specifically. Cell aggregates with multiple cell types have been assembled into defined architectures. These results may provide insights into bottom-up tissue engineering and cell communication.

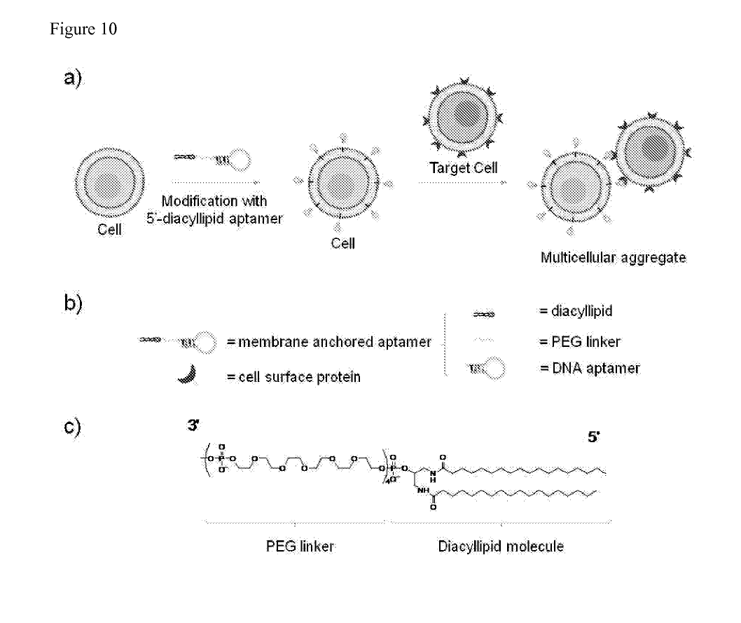

Living organisms have complex cell-cell and cell-environment interactions. Cells adhere to each other and to their extracellular matrix through cell-surface proteins called cell adhesion molecules (CAMs). (Becerle et al. 2002). In fact, orderly tissue architecture is constructed and maintained by selective adhesion of multiple populations of cells. Thus, the ability to control the cell-cell adhesion of multiple cell types is highly desired for cell delivery, tissue engineering and tissue regeneration. In response, techniques, such as layer-by-layer printing, (Ringeisen et al. 2006) optical tweezers (Jordan et al. 2005; Nahmias et al. 2006) and dielectrophoresis methods, (Chiou et al.) have been explored to build a variety of micro-scale 2-D or 3-D assemblies. However, for practical purposes, these methods are limited because they lack the intrinsic molecular encoding for cell-cell recognition. Thus, selective cell assembly from bottom-up approaches remains an important challenge. Due to their exquisite molecular recognition property, synthetic DNA molecules have been programmed to direct the assemblies of a range of nano/micro scale structures. (Mirkin et al. 1996; Alivasatos et al. 1996; Chen et al. 2007; Yoshina-Ishii et al. 2003; Stengel et al. 2007; Yang et al. 2008; Lin et al. 2006). Successful applications of DNA encoding of a wide variety of materials, including inorganic particles, (Mirkin et al. 1996; Alivasatos et al. 1996; Chen et al. 2007 soft materials (Yoshina-Ishii et al. 2003; Stengel et al. 2007; Yang et al. 2008; Lin et al. 2006 and even live cells, (Chandra et al. 2006; Borisenko et al. 2009; Gartner et al. 2009) have been reported for the application in biodiagnostics and tissue engineering. For example, Bertozzi and coworker reported the assembly of 3-D microtissues via DNA hybridization. (Gartner et al. 2009). A main drawback of using DNA hybridization for cell assembly is that it requires the surface modification of target cells. Ideally, it is desired that the modified cells could recognize their target cells spontaneously, without manipulation of target cells. The present work describes a strategy for controlling cell-cell adhesion which mimics the natural process of cell-cell adhesion based on the hypothesis that aptamers would induce cellular adhesions upon receptor-ligand binding. The method of using aptamers to mediate the assembly of cell-cell adhesion is illustrated schematically in FIG. 10. First, the cell surface was modified by incubating the cells with a membrane-anchored aptamer sequence. Then, cell assembly was achieved by incubating the aptamer-modified cells with the target cells. The present work demonstrates that aptamers anchored on the cell surface can act as artificial CAMs that recognize their target cells specifically.

A key step in this strategy calls for facile DNA modification of the live cell surface that enables rapid, efficient and controlled formation of specific 3-D interactions for a range of cell types. The idea of noncovalent cell surface modification has been demonstrated by using a membrane anchored DNA. (Yoshina-Ishii et al. 2003; Stengel et al. 2007; Yang et al. 2008; Lin et al. 2006; Borisenko et al. 2009; Liu et al.; Shea et al. 1990; Chan et al. 2008; Bunge et al. 2007). Compared with covalent methods, the noncovalent method is fast, simple, nondestructive and nontoxic. The membrane anchored DNA can be divided into three distinct segments (FIG. 10 b). The first segment is a cell based aptamer sequence selected by a process called cell-systematic evolution of ligands by exponential enrichment (cell-SELEX). (Shangguan et al. 2006; Tang et al. 2007). Two different aptamers, Sgc8 which specifically targets CCRF-CEM cells, (Shangguan et al. 2006) and Tdo5 which targets Ramos cells, (Tang et al. 2007) are used for the testing. These aptamer sequences exhibit high affinity (K_{dsgc8}: 0.8 nM, K_{dtdo5}: 74 nM) and excellent selectivity toward their targets, both are key elements required for mimicking native cell-surface ligand-receptor interactions. The second segment is a PEG linker. In our design, the neutral PEG molecules help the DNA to stretch out the cell surface, minimizing the charge-charge, nonspecific and steric interactions between the cell-surface molecules and DNA. Thus, the PEG linker facilitates the conformational folding of the aptamer, which is important for aptamer-target interactions. Finally, the third segment, a synthetic diacyllipid tail was conjugated at the 5′ end as the membrane anchor. Because of its hydrophobic nature, the diacyllipid tail could firmly insert into the cell membrane with excellent efficiency. As a result, the aptamers anchor on the cell surface and subsequently act as artificial CAMs for cell assembly.

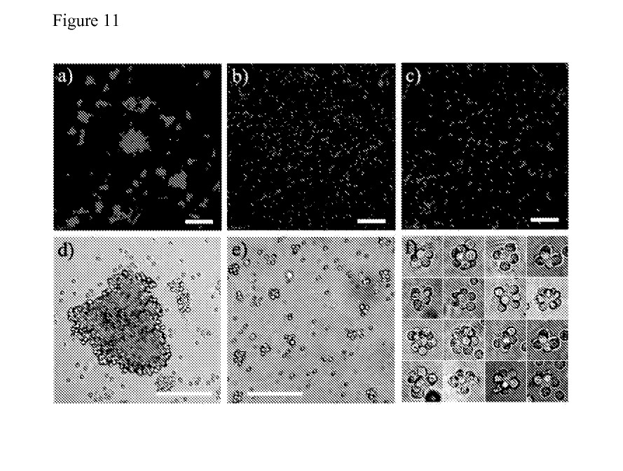

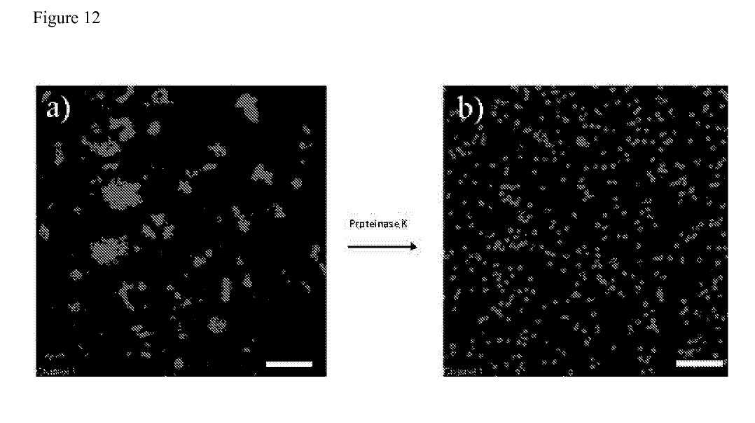

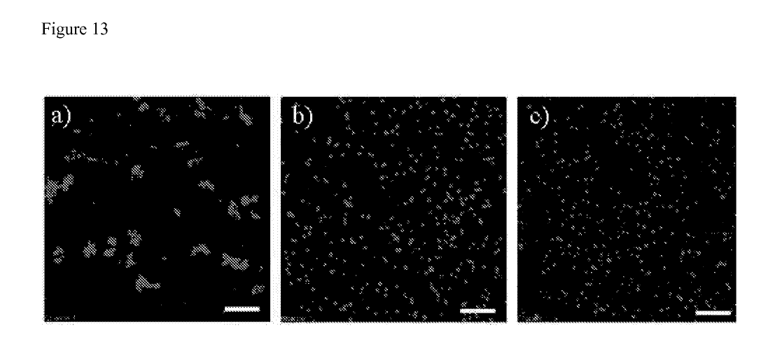

To test the above hypothesis, first a homotypic cell assembly was employed. Suspensions of Ramos cells were modified with TAMRA-labeled aptamer, lipo-Tdo5-TMR, during which time the cells were put on an orbital shaker and incubated at 300 rpm for 30 min. After washing away the free DNA the cells were examined by fluorescence microscopy. The aptamer-treated Ramos cells form large aggregates spontaneously (FIG. 11). Importantly, the formation of aggregates was sequence-specific. In control experiments where either Ramos cells were functionalized with a random sequence (lipo-Lib-TMR) or a negative cell line (CEM) was used, no aggregates were observed (FIG. 11b, c). Finally, when cellular aggregates were treated with proteinase K, a protein known to digest the target surface protein of Ramos cells, (Tang et al. 2007) the aggregated cells dissembled and individual cells were uniformly dispersed (SI, FIG. 12). Similar homotypic assemblies were observed for CEM cells modified with Lipo-sgc8-TMR (SI, FIG. 13). The above experiments demonstrated the hypothesis that membrane-anchored aptamers can induce cellular adhesion in a sequence-specific fashion.

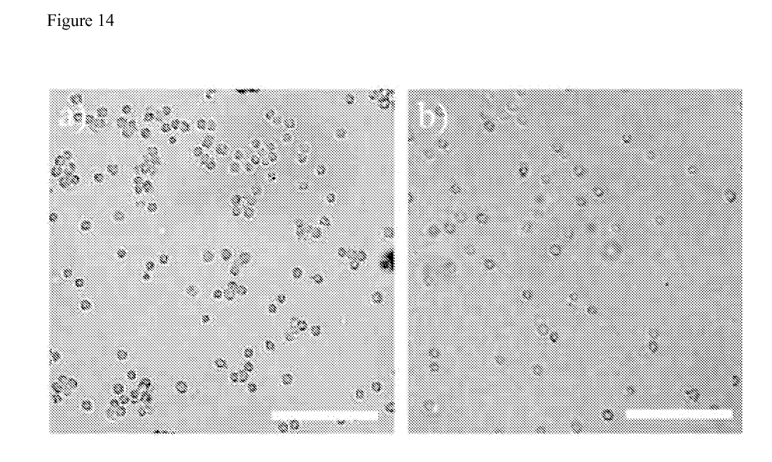

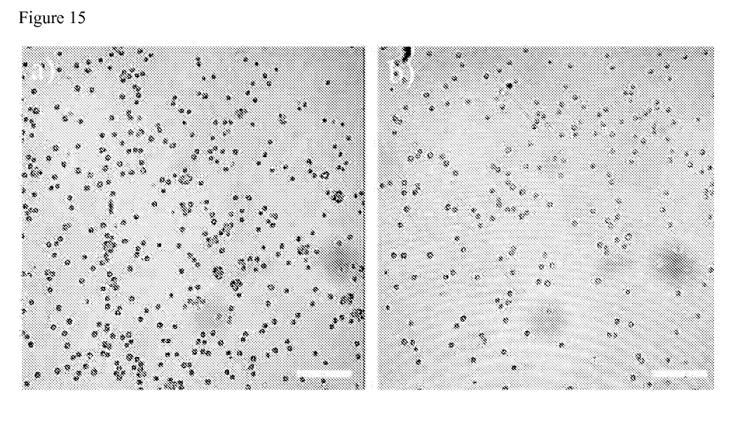

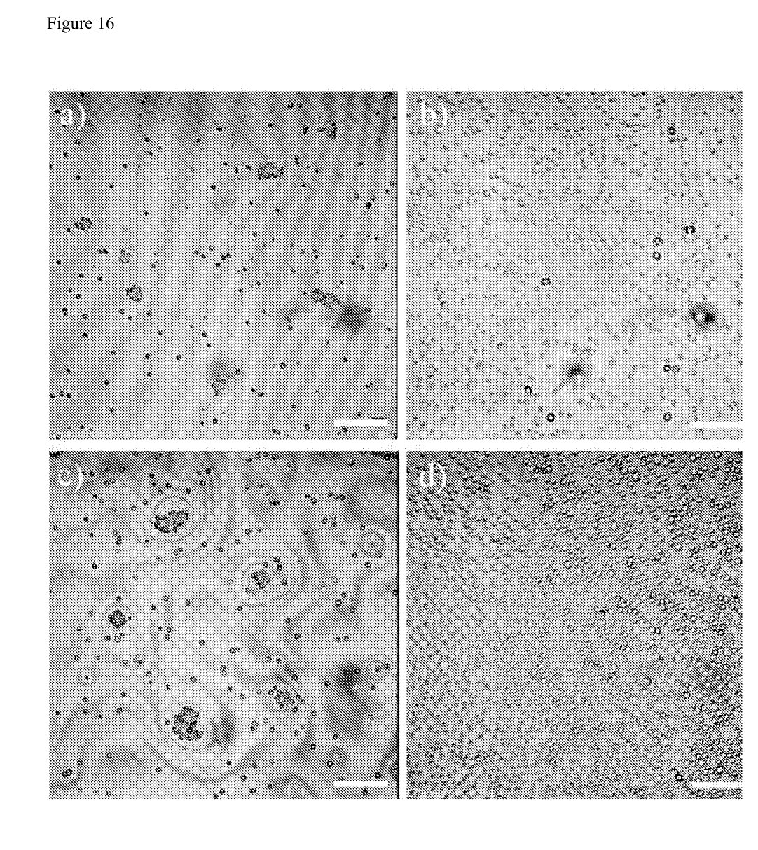

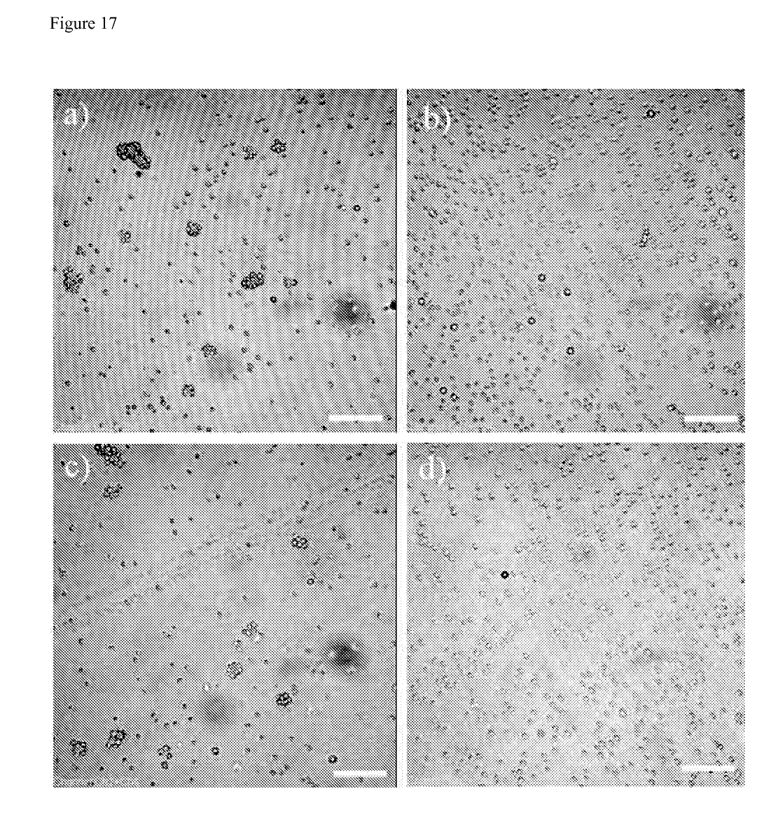

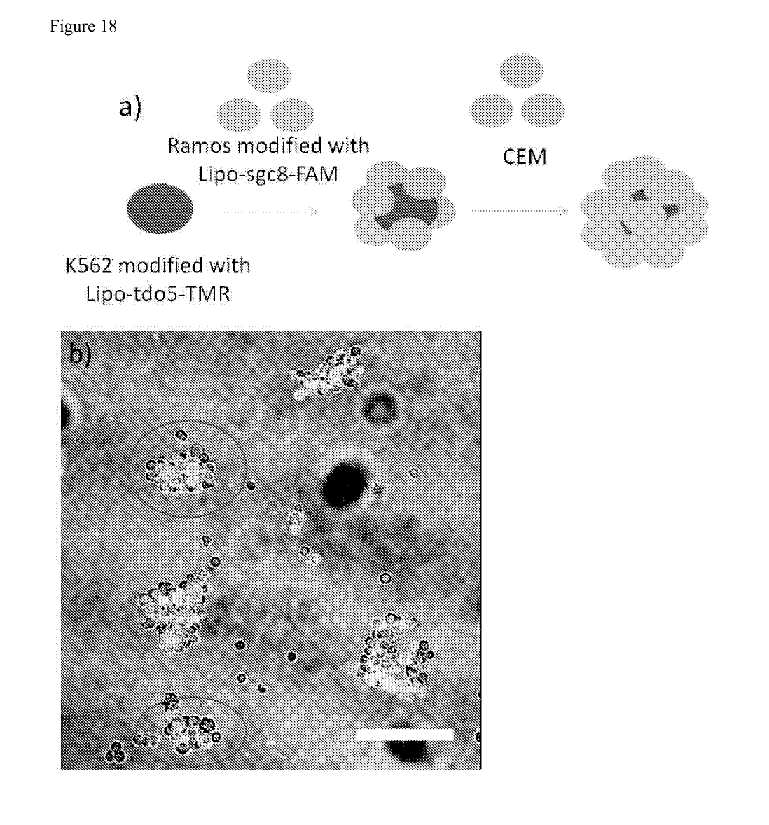

In the next experiments, a heterotypic cell assembly was engineered. Ramos cells were first treated with lipo-Sgc8-TMR, an aptamer sequence for CEM cells. After washing away the free DNA, the modified Ramos cells (fluorescent) were mixed with unmodified CEM cells (nonfluorescent) at 1:1 ratio, and large cell aggregates were observed (FIG. 2d). In order to probe the order of cell-cell interaction, the cell aggregates were vigorously vortexed and subsequently imaged. The large cell aggregates were partially broken down and small clusters were observed (FIG. 14). Each cluster had two types of cells: surface modified fluorescent cells and nonfluorescent target cells. To control the final architecture of the cell assembly, the modified cells were combined and target cells at a ratio of 1:10. (Gartner et al. 2009) Under these conditions individual cell clusters were assembled with defined architecture. As shown in FIG. 11e and 11f, small cell clusters with fluorescent cells surrounded by target cells were observed. Like the homotypic assembly, the heterotypic assembly was formed in a sequence-specific manner (FIGS. 14, 15). Similar heterotypic cell adhesions were also observed between CEM and Ramos cells, using lipo-Tdo5-TMR (FIG. 15). The generality of this assembly method was demonstrated by crosslinking CEM/Ramos with other cell lines such as Jurkat and K562, using similar procedures (SI, FIGS. 16, 17). Finally, this strategy can be extended to engineer 3-D aggregates with more complex structures, as demonstrated in FIG. 18, three different types of cells were sequentially assembled into 3-D aggregates with defined connectivity.

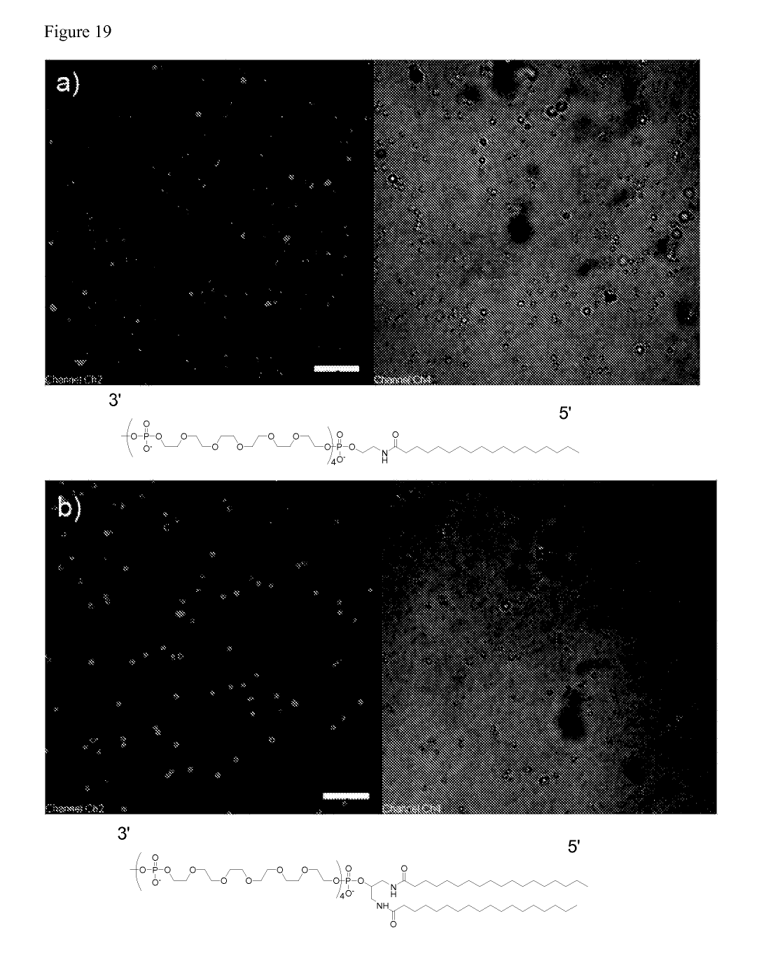

Like the natural cell-cell interactions, multivalent binding is crucial for an effective cell-cell adhesion. When cells were treated with a low concentration of aptamer probes (100 nM, for example), very little or virtually no cell aggregation was observed. At higher aptamer concentrations (>5 μM), cells aggregate within 10 minutes. Despite the high affinity between aptamers and their target proteins, this observation indicates that, multiple receptor-ligand interactions are still required for cell-cell adhesion. This is different from DNA-mediated assembly of nanocrystals, where one copy of DNA is sufficient for their assemblies. (Alivsatos et al. 1996). At the same time, however, the minimum density of aptamers required for assembly, varies according to many factors, including, for example, length of DNA, (Liu et al. 1990) cell type and aptamer affinities. Effective DNA modification on cell also requires a firm membrane anchor. In our experiments, when a monoacyllipid was used, under comparable conditions, low cell fluorescence was observed (FIG. 19). This finding is consistent with a previous report, (Pfeiffer et al. 2004) where bivalent cholesterol has been shown to improve the anchor strength of DNA on lipid membranes.

The present experiments have show the successful engineering of a new type of artificial CAMs composed of membrane anchored aptamer. The present experiments demonstrate the selective assemblies of multiple types of cells via aptamer-protein recognitions. This process is fast, target specific and can be performed under typical cell culture conditions. DNA encoded cell capture and assembly based on DNA hybridization have been reported before, (Chandra et al. 2006; Borisenko et al. 2009; Gartner et al. 2009); however, these methods require tedious procedures for cell-surface modification and the mechanism of cellular recognition is limited to DNA hybridizations. The present strategy is suitably simple, which, in turn, offers several advantages over methods previously reported. First, the cell surface is functionalized by a simple and fast noncovalent modification. Second, the cell recognition is based on specific aptamer-protein interaction, as a result, our method eliminates the need for target cells modifications. Third, the use of aptamers could greatly extend the scope of DNA-encoded cell assembly by taking advantage of the sequence diversity of aptamers. This method could be easily extended to other type of cells, for example, human T cells could be modified with similar method and could be utilized for therapeutic applications. This strategy could also be applied to the study of cell-cell communication, tissue engineering and cancer therapy.

Materials. Unless otherwise stated, all solvents and chemicals were obtained from Sigma-Aldrich without further purification. TMR and FAM labeled CPG beads was purchased from Glen Research. PEG phosphoramidite (DMT-Hexaethyloxy-Glycol phosphoramidite) was purchased from ChemGenes Corporation (Wilmington, Mass.). HPLC was performed on a Varian Prostar system; UV/Vis was recorded by Varian Cary 100 spectrophotometer; 1H NMR and 31PNMR were recorded on a Varian Mercury (300 MHz) spectrometer using tetramethylsilane (TMS) as an internal standard; chemical shifts are reported in ppm (δ) referenced to TMS. The synthesis of lipid phophsphoramidite was followed a previously published procedure.[1-2] Oligonucleotides were synthesized in 1.0 micromolar scale on an automated DNA synthesizer (ABI 3400, Applied Biosystems, Inc.). After cleavage and deprotection with aqueous ammonium hydroxide (55° C., 14 hours), the DNA was purified by reverse phase HPLC and quantified by UV spectrometer.

Synthesis of lipid phosphoramidite. Lipid phophsphoramidite was synthesized by following a previously published procedure. (Liu et al; Gold et al. 2001).

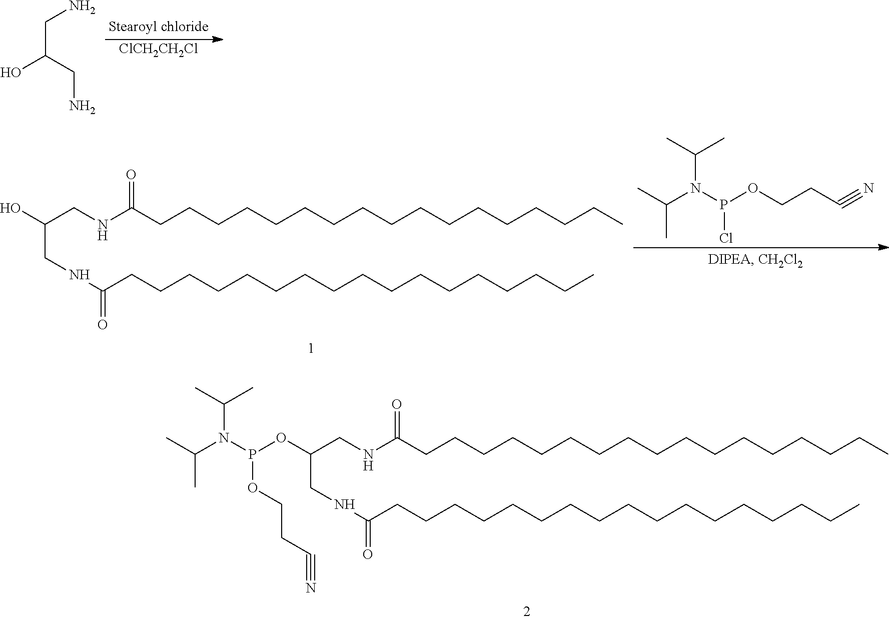

Synthesis of compound 1: A solution of stearoyl chloride (6.789 g, 22.41 mmol) in ClCH₂CH₂Cl (50 ml) was added dropwise to a solution of 1,3-diamino-2-dydroxypropane (1.0 g, 11.10 mmol) in ClCH₂CH₂Cl (100 ml) and TEA (2.896 g, 22.41 mmol), the reaction mixture was stirred for 2 hours at room temperature and 70 degree overnight. The solution was then cooled to RT, filtered, the solid was washed with CH₂Cl₂, CH₃OH, 5% NaHCO₃ and ethyl ether. The solid was dried under vacuum to give compound 1 as white solid (yield, 90%).

Synthesis of compound 2: Compound 4 (5.8 g, 9.31 mmol) was dissolved in anhydrous CH₂Cl₂ (100 ml) and DIPEA (4.2 ml, 18.62 mmol) was injected. The solution was cooled on an ice bath and 2-Cyanoethyl N,N-diisopropylchlorophosphoramidite (8.6 mL, 0.47 mmol) was added under dry nitrogen. After stirred at RT for 1 hour, the solution was heated to 60 degree for 90 minutes. After cooling to RT, the solution was washed with 5% NaHCO₃ and brine, dried over Na₂SO₄ and concentrated in vacuum. The product was purified by precipitation from CH₃CN to afford compound 2 (4 g, 55% yield) as white solids. 31P NMR (CDCl3) 154 ppm.

DNA Synthesis and Sequences. All DNA sequences were synthesized by using the ABI 3400 synthesizer on 1.0 micromolar scale. PEG phosphoramidite was coupled by extended coupling time (900 seconds). Lipid phosphoramidite was dissolved in dichloromethane and was coupled by the so-called syringe synthesis technique. As an alternative, diacyllipid phosphoramidite could also be coupled using the DNA synthesizer. After the synthesis, the DNA was cleaved and deprotected from the CPG and purified by reverse phase HPLC using a C4 column (BioBasic-4, 200 mm×4.6 mm, Thermo Scientific) with 100 mM triethylamine-acetic acid buffer (TEAA, pH 7.5) and acetonitrile (0-30 min, 10-100%) as an eluent. Each DNA probe has coupled four DMT-Hexaethyloxy-Glycol phosphoramidite units.

Aptamer sequences:

Lipo-sgc8-TMR:
5′-Lipid-(PEG)4-T TTT TTT ATC TAA CTG CTG CGC
CGC CGG GAA AAT ACT GTA CGG TTA GA-TMR-3′
Lipo-tdo5-TMR:
5′-Lipid-(PEG)4-A ACA CCG GGA GGA TAG TTC GGT
GGC TGT TCA GGG TCT CCT CCC GGT GA-TMR-3′
Lipo-lib-TMR:
5′-Lipid-(PEG)4-N NNN NNN NNN NNN NNN NNN NNN
NNN NNN NNN NNN NNN NNN NNN NNN NN-TMR-3′

Structures of lipid, PEG and TMR molecules:

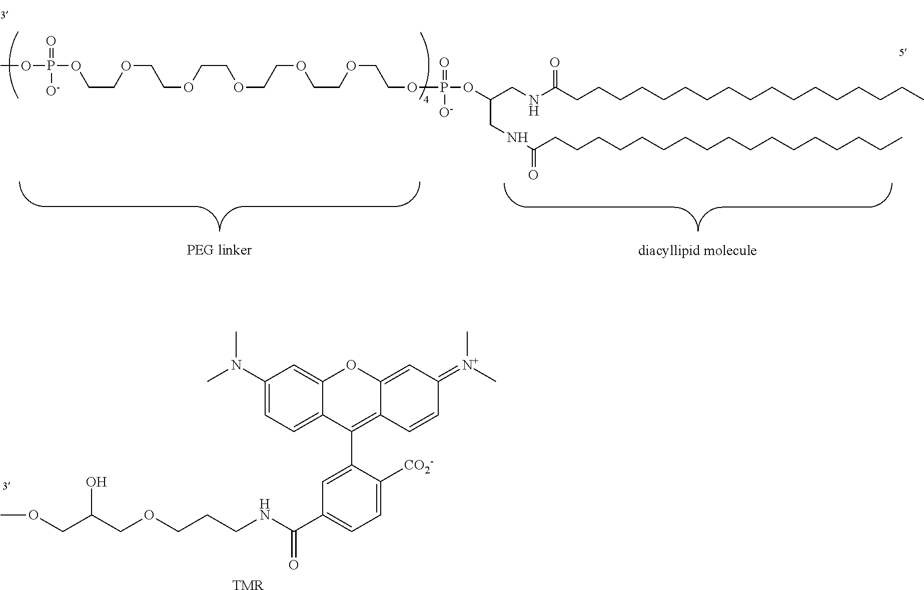

General Cell Culture Conditions. Jurkat, K562, CCRF-CEM (CCL-119 T-cell, human acute lymphoblastic leukemia) and Ramos Cells (CRL-1596, B lymphocyte, human Burkitt's lymphoma) were obtained from ATCC (American Type Culture Collection) and were cultured in RPMI 1640 medium (ATCC) supplemented with 10% fetal bovine serum (FBS) (heat inactivated, GIBCO) and 100 IU/mL penicillin-streptomycin (Cellgro). The washing buffer contained 4.5 g/L glucose and 5 mM MgC12 in Dulbecco's PBS (Sigma). Binding buffer used for incubation was prepared by adding yeast tRNA (0.1 mg/mL) (Sigma) and BSA (1 mg/mL) (Fisher) into the washing buffer to reduce background binding. Proteinase K was purchased from Fisher biotech.

Cell Surface Labeling and Cell Aggregation. For homotypic cell assembly, cells (200 μL, 1×106 cells/mL) were suspended in a 96-well plate, incubated with DNA probe (5 μM lipid-DNA) in binding buffer at 37° C. for 3 hours, then the cells were washed three times with washing buffer, resuspended in binding buffer, and imaged under fluorescence microscope. For heterotypic cell assembly, Cells (200 μL, 1×106 cells/mL) were suspended in a 96-well plate, incubated with DNA probe (5 μM lipid-DNA) in cell culture media at 37° C. for 3 hours, then the cells were washed three times with washing buffer, resuspended in binding buffer, and a proper ratio of cells were combined in binding buffer and shaken at 300 rmp for 30 min at 25° C. Aliquots were analyzed by fluorescence microscopy.

Proteinase Treatment of Cells. After washing with 2 mL of washing buffer, Ramos cells were incubated with 0.1 mg/mL proteinase K in PBS at 37° C. for 20 min. To quench the proteinase digestion, the sample was quickly mixed with 200 μL of PBS and placed on ice. Then the treated cells were washed with 2 mL of binding buffer and used for imaging.

### Example 6

### DNA-Aptamer Micelle as an Efficient Detection/Delivery Vehicle to Cancer Cells

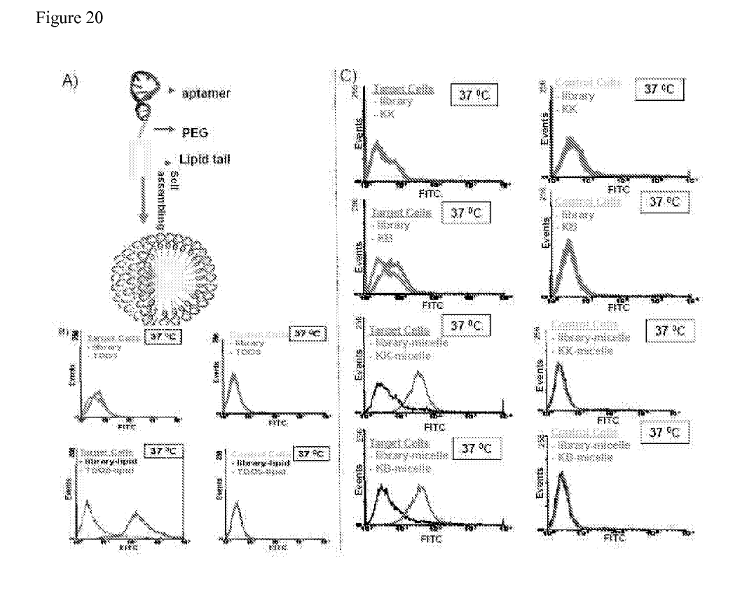

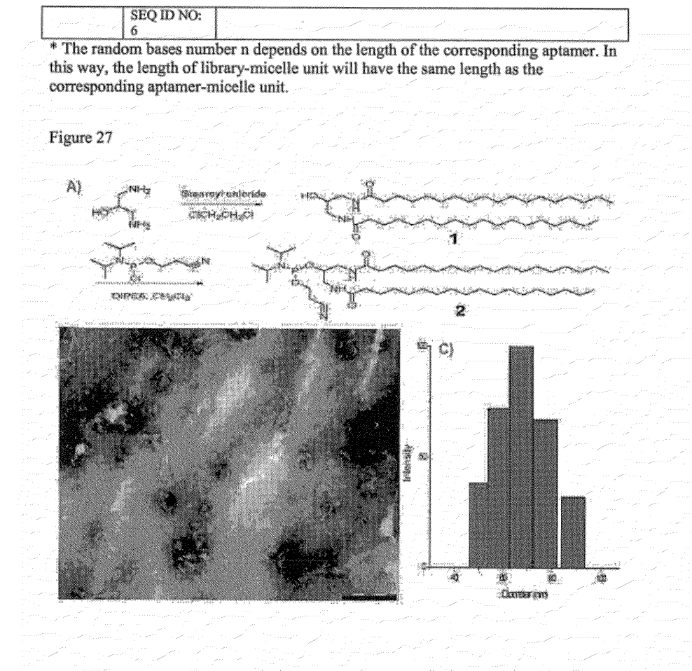

Aptamer-micelle construction. As shown in FIG. 20, a simple lipid tail phosphomidite with diacyl chains was attached onto the end of an aptamer inserted with a PEG linker. This amphiphilic unit self-assembled into a spherical micelle structure, as demonstrated in the TEM image (FIG. 27). The aptamer used in this case is called TDO5, which was selected specific to Ramos cells (a B-cell lymphoma cell line) (Tang, 2007). As shown in FIG. 27B, the TDO5-micelle has an average diameter of 68±13 nm, which is consistent with the hydrodynamic diameter measured by Dynamic Light Scattering of 67.22 nm (FIG. 27C).

Enhanced binding at physiological temperature. Interestingly and surprisingly, the formation of an aptamer-micelle was found to enhance the binding capability of otherwise low-affinity aptamers at physiological temperature. TDO5 is such an aptamer. At 4° C., TDO5 showed high affinity and selectivity for its target protein, IGHM (immunoglobin heavy mu chain) receptor on cell surface (Mallikaraktchy et al. 2007), which indicates that this surface cell membrane protein has an upregulated expression in Ramos cells. On the other hand, TDO5 did not bind with Ramos cells at 37° C. (FIG. 20B). However, when TD05 is used for micelle formation, the TDO5-micelle was also found to have excellent binding selectivity at 37° C. As shown in FIG. 20B, when binding with target cells, about 80-fold enhancement in fluorescence intensity was observed for TDO5-micelle, while no binding shift was found with control cells.

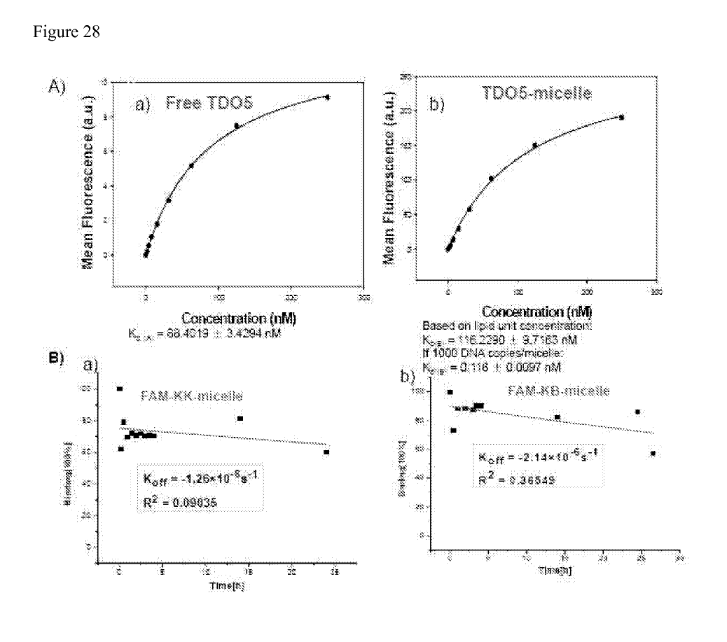

The dissociation constants of the aptamer-micelles were also investigated. Since a similar size of polymer micelle (60 nm) was estimated to have 1000 copies of units (Chen et al. 2008), one TDO5-micelle (68 nm) is assumed to have the same estimated unit numbers. As shown in FIG. 28A, the TD05 micelle has a Kd of 116 nM. If one TDO5-micelle has 1000 copies of DNA-lipid units, the dissociation constant after constructing the micelle structure would be greatly decreased from 88 nM (for free TDO5) to 0.116 nM (for TDO5-micelle). This approximate 750-fold increase in binding affinity might be ascribed to the multivalent effect from multiple aptamer binding.

Based on these findings, the lipid tail plus linker modification can be a universal strategy to promote the binding abilities of low-affinity aptamers, as demonstrated by the improved binding behaviors observed after attaching lipid tails onto the end of two other low-affinity aptamers, KK and KB (FIG. 20C).

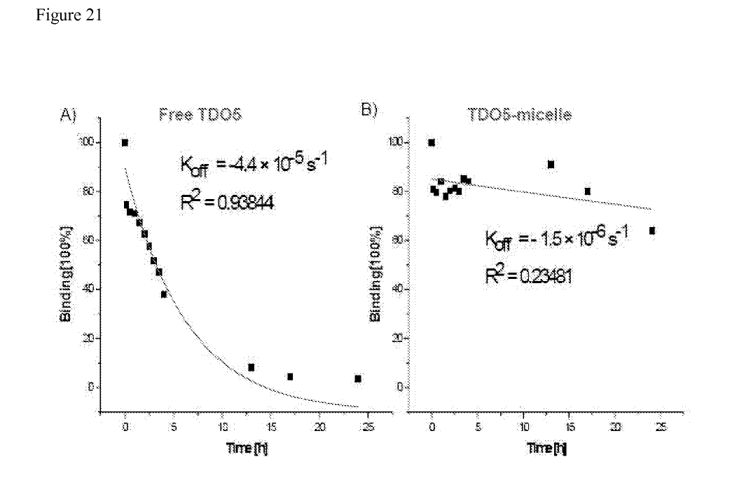

Extremely low K_{off}. Generally, a low off rate tends to indicate that the binding is strong and difficult to be replaced. To test the micelle's binding strength in comparison to single aptamer, the Koff rate of the aptamer-micelle was examined by a competition experiment. As shown in FIG. 21, after one day of competition with unlabeled TDO5 aptamer, almost all the bound labeled TDO5 was replaced. Plotting the competition data of free TDO5 showed an exponential decay with a K_{off} of −4.4×10−5 S−1 (R²=0.93844). In contrast, after the same competition condition, a very low percentage of bound TDO5-micelles was replaced by free TDO5 with a K_{off} of −1.5×10−6 S−1 (R²=0.23481). It was noted that the coefficient for the data fitting in TDO5-micelle case is quite low, which indicates that the off rate pattern of TDO5-micelle might not be the same as the exponential pattern. Similar low off rates and low R² were observed for the other two aptamer-micelles based on KK and KB aptamers (FIG. 28B).

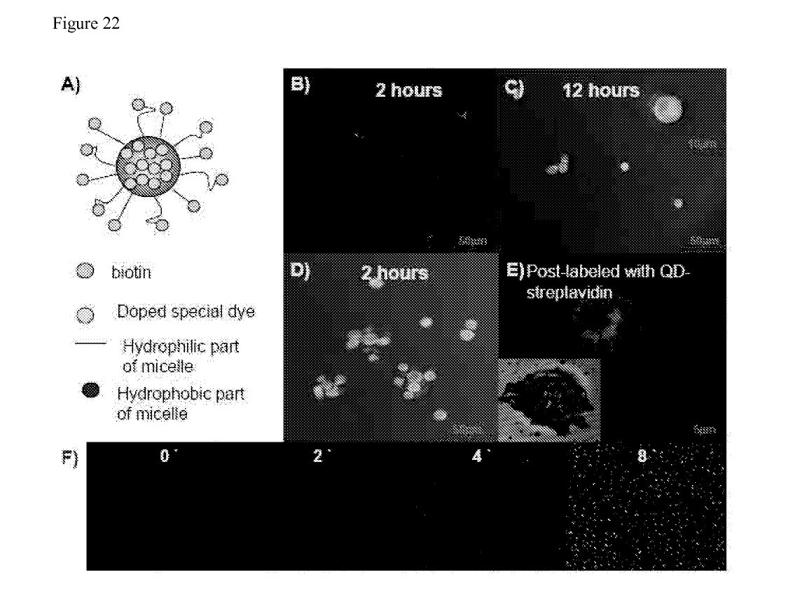

Based on these extremely low off rates, considering the fact that both aptamer-micelle and cell membrane have hydrophobic and hydrophilic portions, it was speculated that the aptamer-micelle can be integrated into the cell membrane facilitated by the lipid tail and that the preferred thermal stability does not allow the aptamer-micelle to easily diffuse out. In order to determine whether the aptamer-micelles could fuse with cell membrane, the TDO5-micelles were doped with a special dye that only fluoresces inside cells (CELL TRACKER) (see scheme in FIG. 22A). As shown in FIGS. 22B and 22C, after incubating the cells with free cell tracker for 12 hours, a strong fluorescence signal was observed inside the cells, while a very weak fluorescence signal was present after 2 hours of incubation. In contrast, it was only after an incubation of 2 hours with dye-doped TDO5-micelles that most of the cells produced a strong fluorescence signal (FIG. 22D). To determine whether some or all the aptamer-micelles remained on the cell surface, streptavidin-quantum dots 705 (QD705) were post-incubated with the cells after binding biotin-labeled TDO5-micelle to the cells. FIG. 22E shows a strong red fluorescence signal around the cell membrane, which indicates that at least some of the aptamer-micelles remained bound to the cell membrane after the dye was released. Exposing the cells to strong UV illumination for a long time leads to cell apoptosis and the leakage of activated fluorescent cell tracker dyes into the incubation buffer. As shown in FIG. 22E, a strong green fluorescent signal was observed outside the apoptotic cells while a clear QD705 halo remained where the cell membrane would have been, indicating the integration of the aptamer-micelle into the membrane (FIG. 22E inset). Based on real-time monitoring of the fluorescence from the cell tracker at room temperature (FIG. 22F), fusion of the micelles with the cell membranes occurs within minutes.

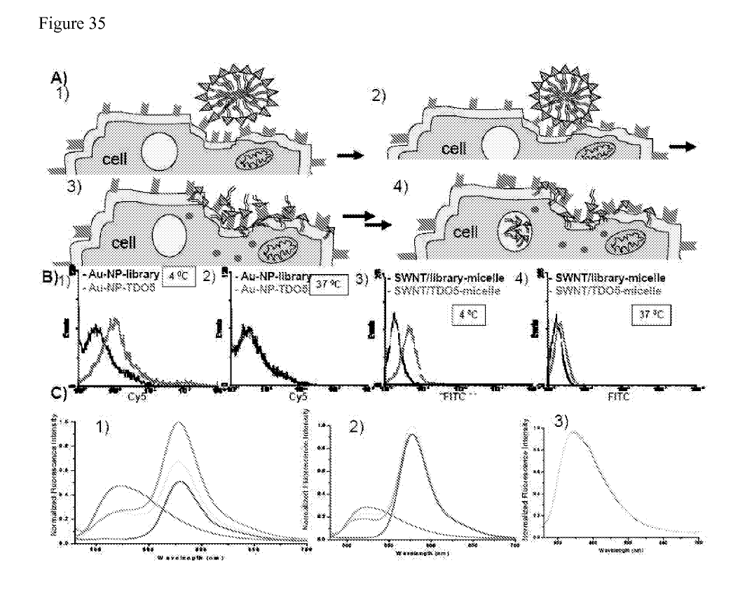

The above experiments reveal the potential fusion between aptamer-micelles and the cell membrane. Thus, the interaction process between aptamer-micelles and cells is speculated to be fluidic in nature, involving specific interaction induced nonspecific insertion (see Supporting Information FIG. 35 for detailed hypothesized mechanism and related experiments).

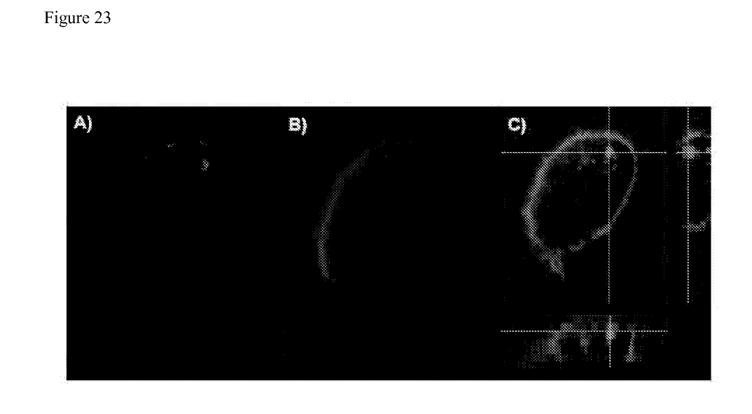

TDO5-micelle helps cell internalization. Although some of the aptamers by themselves lack an internalization pathway, the introduction of this novel nanostructure formation allows the aptamer-micelles to ultimately penetrate the cells they target. As shown in FIG. 23, a clear fluorescence signal inside the cells confirmed by optical imaging with confocal Z-axis depth scanning was observed. Since TDO5 alone does not internalize, there must be another mechanism occurring, which might be related to membrane recycling. This interesting internalization pathway created by the attachment of the lipid tail, as detailed above, can widen aptamer applications in therapy which usually requires conjugated drug delivered into the cells.

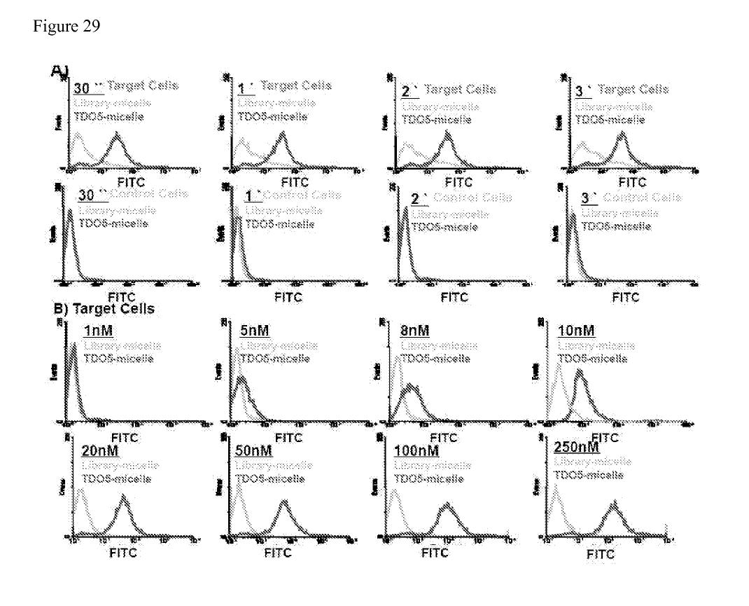

Rapid identification with high sensitivity. This TDO5-micelle demonstrated extremely rapid recognition of the target cells. As shown in FIG. 29A, after an incubation of only 30 sec at 37° C., a 45-fold enhancement in fluorescence intensity when binding with target cells was observed for TDO5-micelle. Again, no significant binding was observed for the control cells. Since every piece of DNA-lipid is labeled with one single FITC dye at the 3′ end, one recognition event from one aptamer-micelle can induce multiple-dye staining to target cells. Therefore, this aptamer-micelle structure is suggested to provide an additional signal enhancement. As shown in FIG. 29B, even at about 0.005 nM (or 5 nM, based on DNA-lipid concentration), noticeable fluorescence shift was still observed when binding with target cells.

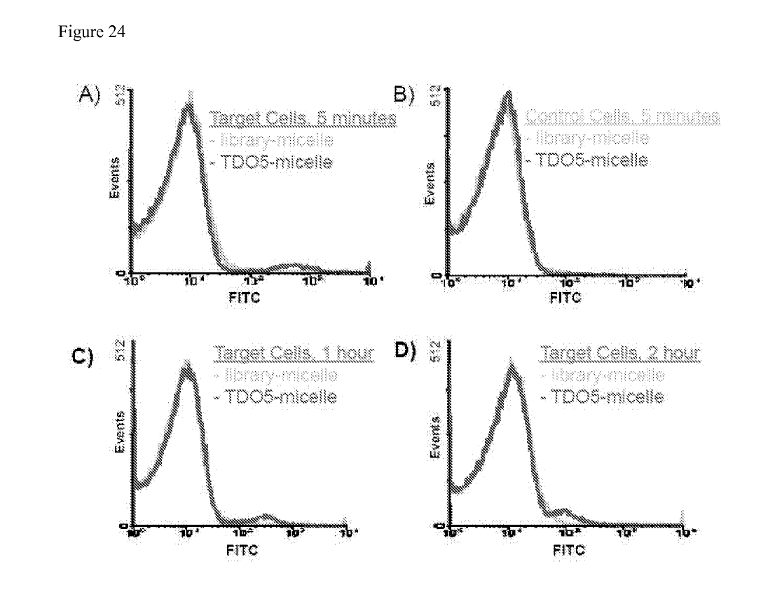

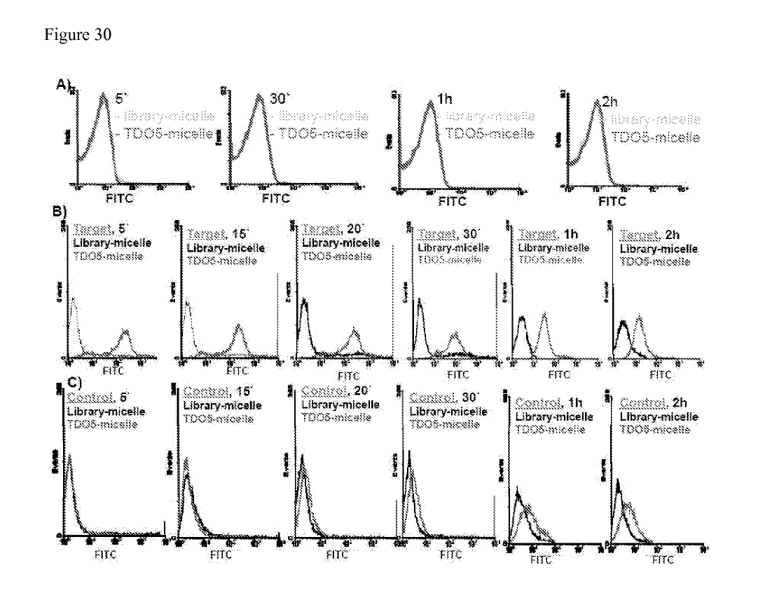

Trace cell detection in whole blood sample. To evaluate the detection ability of TDO5-micelle in a complex environment, one million target cells/control cells were spiked directly in 50 uL human whole blood sample (about 310 million cells) and then the DNA-micelles were incubated with the cell mixture at 37° C. for 5 minutes. Based on the flow data shown in FIG. 24, obvious binding shift was observed when binding to the target cells, but no significant binding shift happened in the control cell mixture. Similar to control, no binding shifts happened in the absence of spiked target cells in whole blood sample (see FIG. 30A). These flow data prove that the aptamer-micelle can detect trace target cells selectively, even in a complex environment.

Meanwhile, when the incubation time was lengthened from 5 minutes to 2 hours, smaller binding shifts were observed (FIG. 24CD). It is suggested that the susceptibility of DNA to enzyme digestion in plasma at 37° C. after long incubation is the main reason for the reduced binding shifts (Tsui et al. 2002). The nuclease digestion of DNA probes has been investigated before and there are modifications one can do to increase the stability of these DNA aptamers (Yang et al. 2007; Shangguan et al. 2007).

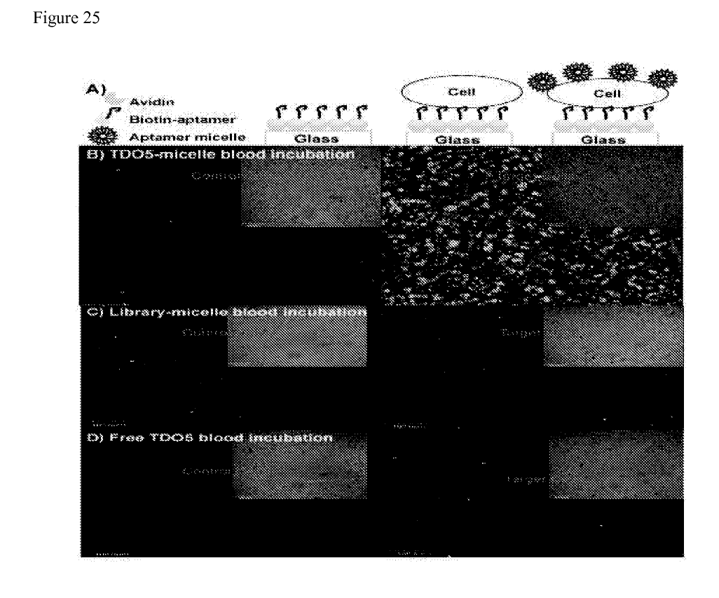

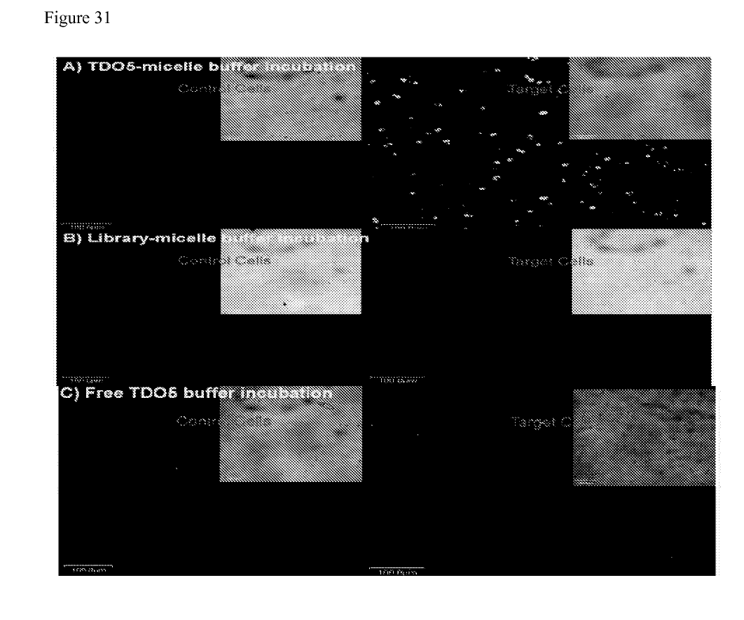

It is not believed that the higher nonspecific binding of DNA-micelles after long incubation in this complex environment causes the smaller binding shifts. As shown in FIGS. 25 and 31, the nearly identical fluorescence intensities of library-micelle were observed irrespective of how long it was incubated with whole blood cell mixture. Comparing the increased nonspecific bindings with increased incubation time in pure buffer sample, as shown in FIG. 30B, one possibility is that the difference in total cell numbers in these two different cases may well lead to different nonspecific interaction patterns. Since the total cell number is extremely high per whole blood cell mixture sample (about 311 million cells), but much lower for the pure buffer incubation (only 1 million cells), the micelle concentration per cell should be extremely low. This leads to the absence of nonspecific binding, even after 2 hours of incubation for whole blood cell mixture.

Aptamer-micelle targeting in a flow channel under a continuous flow. To investigate whether aptamer-micelles can be used for selective targeting under the dynamic fluid conditions of blood circulation, a simplified flow channel was used to mimic the circulatory environment. While there is a considerable difference between the fluidic channel and the blood vessel, it is thought that this flow dynamic study will provide information about future possibilities in using this micelle system as a detection/delivery system with dynamic specificity. As shown in the scheme in FIG. 25A, two biotin-labeled aptamers (biotin-sgc8 and biotin-TDO5) were individually immobilized onto either side of a glass channel using avidin-biotin interactions. Following this step, their corresponding target cells (CCRF-CEM for sgc8 and Ramos for TDO5) were flowed through and captured by the immobilized aptamers. In this way, two different cell zones were established in the flow channel: CCRF-CEM in the control cell zone and Ramos in the target cell zone for TDO5 aptamer.

As the first step, the targeting ability of aptamer-micelle spiked in a simple pure binding buffer system at 37° C. inside the flow channel under continuous flushing was evaluated. For micelle-buffer incubation, either FITC-TDO5-micelle or FITC-library-micelle diluted in binding buffer was continuously flushed through the two cell zones sequentially at 37° C. for 5 minutes at 300 nL/s. The same results were observed irrespective of which direction the DNA-micelle was sucked through the two types of cell zones. A representative result after micelle-buffer incubation with FITC-TDO5-micelle is shown in FIG. 31. In this case, strong fluorescence signal was seen from the target cell zone, but no noticeable fluorescence signal from the control cell zone. In contrast, no fluorescence signal was observed in either cell zone after incubating with a control FITC-library-micelle or free FITC-TDO5 aptamer at 37° C.

As the second step, blood circulation in living systems was mimicked to further test the targeting ability of aptamer-micelle spiked in complex human whole blood sample under continuous flushing at 37° C. To avoid cleaning difficulties, a simplified flow channel made of double glass slides was preferred over a PDMS flow channel for micelle-blood incubation. Representative results after micelle-blood incubation are presented in FIG. 25. As in the micelle-buffer incubation system, strong fluorescence signal was seen from the target cell zone after incubation with TDO5-micelle spiked in whole blood sample, but no noticeable fluorescence signal from control cell zone was seen. In contrast, no fluorescence signal was observed in either cell zone after incubating with a control FITC-library-micelle spiked in whole blood sample.

Although circulation velocity in living systems might be much faster than 300 nl/s in most locations (Duffy et al. 1998), it would be expected that the aptamer-micelle have recognition to the target cells better than, or at least equal to, that which is shown in this dynamic incubation channel with a faster circulation rate. Based on the in vitro study of the effect of the incubation time on cell recognition ability using fixed cell numbers (FIG. 31B), increasing incubation time resulted in a decrease of selectivity; thus, a shorter incubation time might either have no effect at all, or might even lead to better binding selectivity, especially considering the rapid identification ability of aptamer-micelles. These results indicate that the aptamer-micelle can perform selective recognition in a complex environment that mimics (Duffy et al. 1998) blood circulation.

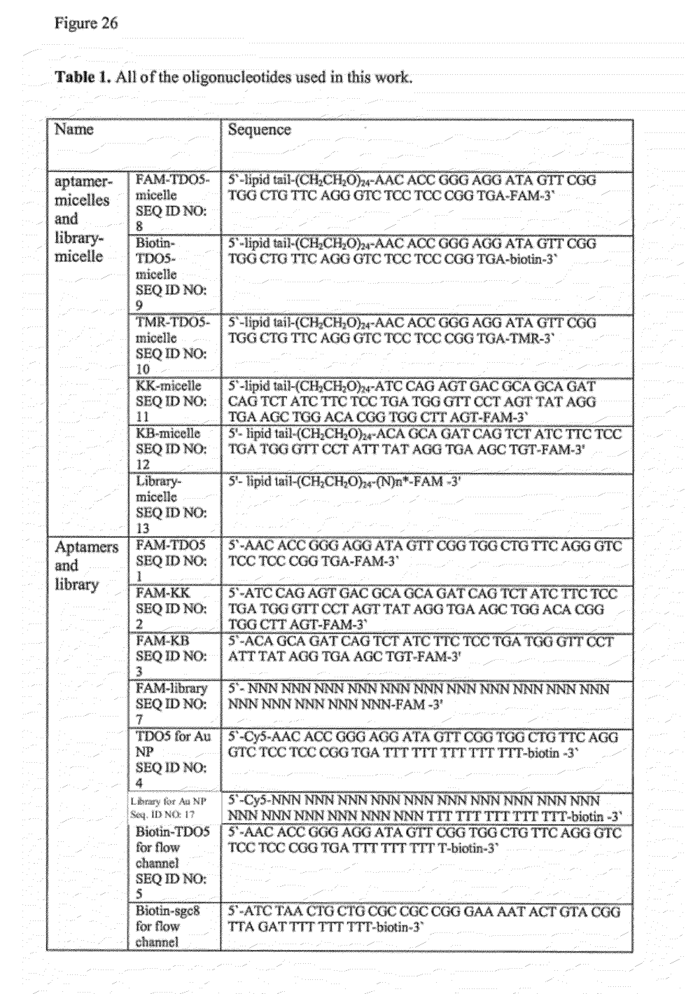

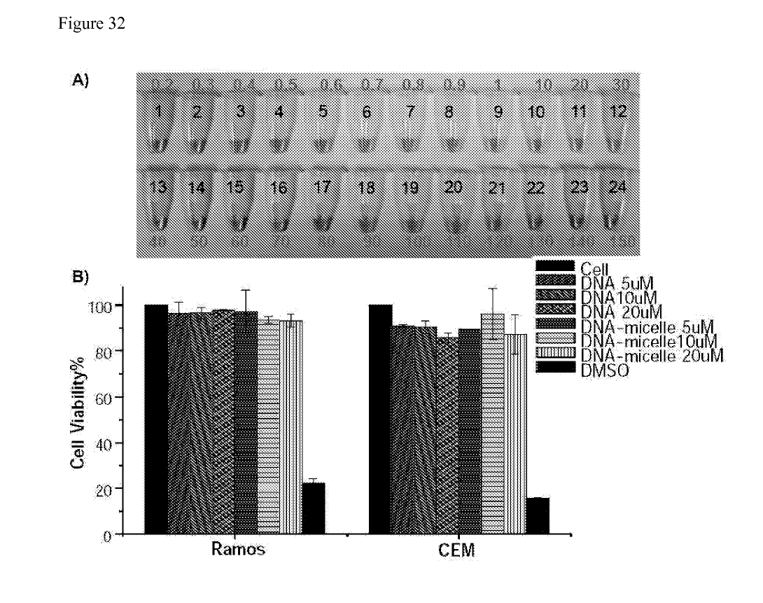

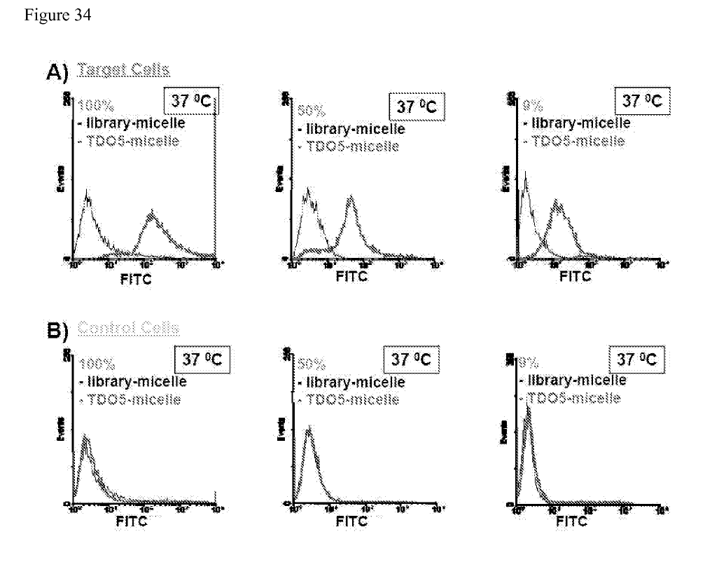

Moreover, this aptamer-micelle was found to have low CMC and low cytotoxicity to the cells (FIG. 32). As such, this type of aptamer-micelle is proposed to be an efficient drug delivery vehicle for target cells without the need for internalization of the aptamer's target molecule. Instead, aptamer-micelles can simply interact with the cell membrane and quickly release the doped hydrophobic molecules into the cells, which is similar to the published polymeric micelle system (Chen et al. 2008). Meanwhile, however, through membrane replacement, aptamer-micelles permeate cells. Thus, this type of aptamer-micelle offers two kinds of drug delivery pathways: direct releasing of doped drug and internalization by direct drug-aptamer-micelle conjugation. Finally, by replacing PEG-lipid with therapeutic aptamer drug-lipid, the heterogeneous aptamer-micelle can specifically deliver aptamer drugs around the target cell surface. For instance, by lipid tail plus linker modification, we can construct a lipid molecule from Macugen, an FDA-approved aptamer selected against vascular endothelial growth factor (VEGF). By replacing PEG-lipid with Macugen-lipid in FIG. 34, it is expected that this aptamer-micelle will be able to draw all the Macugen-lipids to a specific tumor cell surface, which would greatly increase the localized drug concentration to enhance inhibition potency. FIG. 26 is a list of aptamers that were employed in these experiments.

In summary, the present experiments and data show the development of an aptamer-micelle assembly for efficient detection/delivery targeting specific cancer cells. This aptamer-micelle enhances the binding ability of the aptamer moiety at physiological temperature, even though the corresponding free aptamer loses its binding ability under the same condition. The merits of aptamer-micelles include greatly improved binding affinity, low K_{off} once on the cell membrane, rapid targeting ability, high sensitivity, low CMC values, and the creation of a dual drug delivery pathway. Most importantly, the aptamer-micelles show great dynamic specificity in flow channel systems that mimic drug delivery in a flowing system. All of these advantages endow this unique assembly with the capacity to function as an efficient detection/delivery vehicle in the biological living system.

### Methods

The invention was performed using, but not limited to, the following methods and materials.

Unless otherwise stated, all solvents and chemicals were obtained from Sigma-Aldrich without further purification. HPLC was performed on a Varian Prostar system; UV/Vis was recorded by Varian Cary 100 spectrophotometer; fluorescence spectra were obtained on SPEX® FluoroLog® fluorometer; 1H NMR, 31PNMR were recorded on a Varian Mercury (300 MHz) spectrometer using tetramethylsilane (TMS) as internal standard; chemical shifts are reported in ppm (δ) referenced to TMS. Oligonucleotides were synthesized in 1.0 micromolar scale on an automated DNA synthesiszer (ABI 3400, Applied Biosystems, Inc.). After cleavage and deprotection with aqueous ammonium hydroxide (55° C., 14 hours), the DNA was purified by reverse phase HPLC and quantified by UV spectrometer. For the DNA-Aptamer experiments, DNA synthesis reagents were purchased from Glen Research (Sterling, Va.). The single-walled carbon nanotubes (SWNTs) were purchased from Unidym, Inc. with <5 wt % ash content (CAS number: 7782-42-5). CellTracker™ Green BODIPY (C2102) and Qdot 705 streptavidin conjugate (Q10161MP) were purchased from Invitrogen. All flow cytometry data were acquired with a FACScan cytometer (Becton Dickinson Immunocytometry Systems, San Jose, Calif.). Ramos (CRL-1596, B-cell line, human Burkitt's Lymphoma), CCRF-CEM (CCL-119, T-cell line, human Acute Lymphoblastic Leukemia), K562 (CCL-243, chronic myelogenous leukemia (CML), and HL60 (CCL-240, acute promyelocytic leukemia) were obtained from ATCC. The NB4 cell line was kindly provided by Shands Hospital. All cell lines were cultured in RPMI 1640 medium (ATCC) supplemented with 10% fetal bovine serum (FBS) (heat inactivated, GIBCO) and 100 IU/mL penicillin-streptomycin (Cellgro). The wash buffer contained 4.5 g/L glucose and 5 mM MgCl₂ in Dulbecco's PBS (Sigma). Binding buffer used for the aptamer binding assays was prepared by adding yeast tRNA (0.1 mg/mL) (Sigma) and BSA (1 mg/mL) (Fisher) into the wash buffer to reduce background binding.

DNA synthesis. All DNA sequences were synthesized by using the ABI 3400 synthesizer on 1.0 micromolar scale. Pyrene phosphoramidite was coupled by extended coupling time (900 seconds). Lipid phosphoramidite was coupled by the so-called syringe synthesis technique.1 After the synthesis, the DNA was cleaved and deprotected from the CPG and purified by reverse phase HPLC using a C4 column (BioBasic-4, 200 mm×4.6 mm, Thermo Scientific), 100 mM triethylamine-acetic acid buffer (TEAA, pH 7.5)-acetonitrile (0-30 min, 10-100%) as an eluent.

Agarose Gel Electrophoresis. Each DNA sample (1 μg) was analyzed by electrophoresis for about 90 min, under constant 75 V, through a 4% agarose gel in TBE Tris(hydroxymethyl)aminomethane (Tris, 89 mM), ethylene diamine tetraacetic acid (EDTA, 2 mM), and boric acid (89 mM), pH 8.0.) buffer. The bands were visualized by UV illumination (312 nm) and photographed by a digital camera.

Critical Micelle Concentration determination. Typically, amphiphilic DNA was diluted in series concentrations using PBS buffer (120 μL). An equal volume of acetone was added to the solutions so that the excimer fluorescence disappeared. The acetone was evaporated by a speed vacuum, and the volume of the solution became about 50 μL. The solutions were then diluted to 120 μL, and fluorescence spectrum was measured by a fluorometer. The critical micelle concentration was determined by the distinguishable pyrene excimer fluorescence of the corresponding DNA concentration.

Micelle Characterization. AFM images were obtained using a Nanoscope IIIa (Digital Instruments) operated under tapping mode. A drop of DNA sample solution (2 μL) was spotted onto freshly cleaved mica (Ted Pella, Inc.) and left to adsorb to the surface for 30 seconds; then, 1×PBS buffer (50 μL) was placed onto the mica. Imaging was performed by tapping mode AFM under PBS buffer in a fluid cell, using NSC18/ALBS tips (Silicon cantilever, MikroMasch, Inc.) The tip-surface interaction was minimized by optimizing the scan set-point.

The particle sizes (diameters) and their distribution were measured by a ZetaPALS DLS detector (Brookhaven Instruments, Holtsville, N.Y., USA) at 25° C. The scattering angle was fixed at 90°.

For the DNA-Aptamer experiments, micelle characterization was performed as follows TEM images were obtained after negative staining with 1% aqueous Uranyl Acetate using a transmission electron microscope (Hitachi H-7000). The TEM samples were dropped onto standard holey carbon-coated copper grids. Dynamic Light Scattering measurements were carried out at room temperature using a Brookhaven ZetaPALS analyzer. Aptamer-micelles were dissolved in PBS buffer using a transmission electron microscope (Hitachi H-700).

CEM cell culture. CEM cells were grown in Dulbecco's modified Eagle's medium supplemented with 10% fetal bovine serum (Invitrogen) at 37° C. in 5% CO2. Cells were cultured in a 96-well plate, incubated with DNA micelles, and imaged using scanning confocal microscopy.

Preparation of Small Unilamellar Vesicles (SUVs). Stock lipids (1,2-Distearoyl-sn-glycero-3-phosphocholine, DSPC) were dried from chloroform solution under nitrogen and then under vacuum overnight. The lipids were resuspended in a 1×PBS solution, pH 7.4, by vortex mixing. The lipid suspension was then passed through two stacked polycarbonate filters (100 nm) nineteen times in a mini-extruder (Avatilipids).

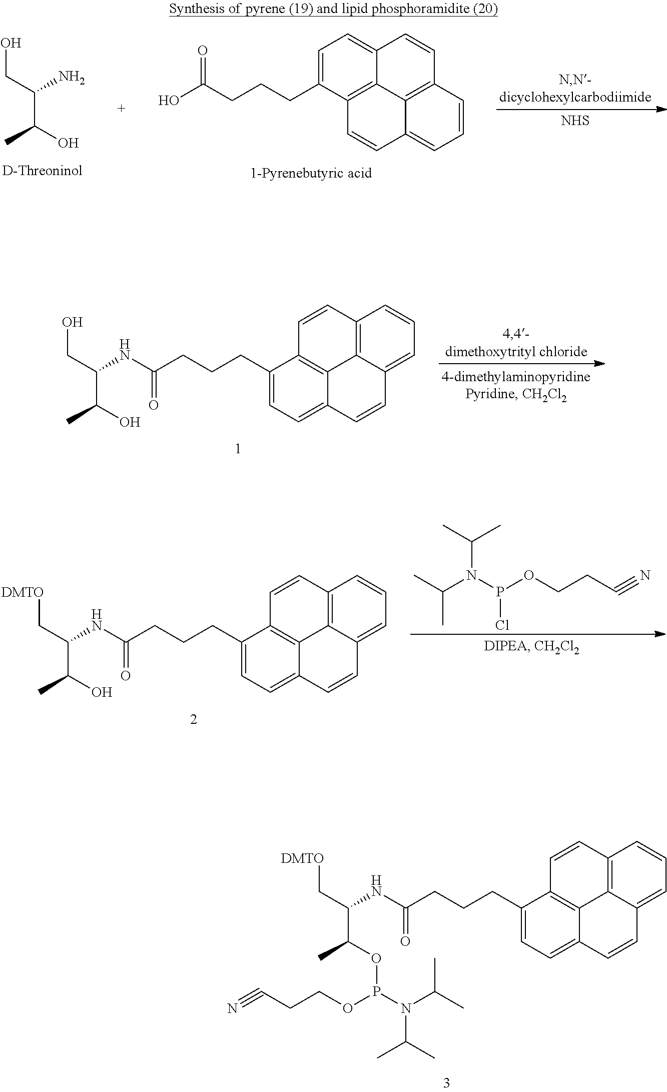

Synthesis of compound 1: In a 300 ml round-bottom flask, D-threoninol (0.95 g, 9.1 mmol), 1-Pyrenebutyric acid (2.88 g, 10.0 mmol), DCC (2.06 g, 10.0 mmol) and NHS (1.15 g, 10 mmol) were dissolved in 50 ml DMF. The reaction mixture was stirred at room temperature for 24 hours. The insoluble N,N′-dicyclohexylurea was filtered, and DMF was removed with a rotary vacuum evaporator to obtain an oily crude product. Compound 1 was purified by flash chromatography (yield: 85%).

Synthesis of compound 2. Compound 1 (2.93 g, 7.2 mmol) and 4-dimethylaminopyridine (0.043 g, 0.36 mmol) in 40 ml dry pyridine in a 100 ml round-bottom flask under dry nitrogen. The solution was cooled on an ice bath. DMT-C1 (2.93 g, 8.64 mmol) was dissolved in 10 ml dry CH2Cl2 in a 50 ml flask under nitrogen and slowly added to the above pyridine solution under dry nitrogen. The reaction was slowly warmed up to room temperature and stirred for 24 hours. The solvent was removed under vacuum, and compound 2 was isolated by chromatography (50:50:3 ethyl acetate:hexane/triethylamine) (yield: 75%).

Synthesis of compound 3. Compound 2 (1 g, 1.48 mmol) was dissolved in CH2Cl2 and cooled on an ice bath ; then, DIPEA (0.57 g, 4.44 mmol) and 2-Cyanoethyl N,N-diisopropylchlorophosphoramidite (0.42 g, 1.78 mmol) were added under dry nitrogen. The reaction mixture was stirred on ice for 3 hours. The solvent was evaporated, and compound 3 was purified by chromatography (50:50:3 ethyl acetate:hexane/triethylamine). Yield: 70%; ¹H NMR (300 MHz, CDCl3): δ 8.3-6.6 (m, 21H), 5.82 (d, 1H), 4.4-4.2 (m, 2H), 3.8 (s, 3H), 3.7 (d, 6H), 3.6-3.1 (m, 8H), 2.5 (m, 1H), 2.4-2.2 (m, 5H), 1.3-0.9 (m, 20H). ³¹P NMR (CDCl3) 149.

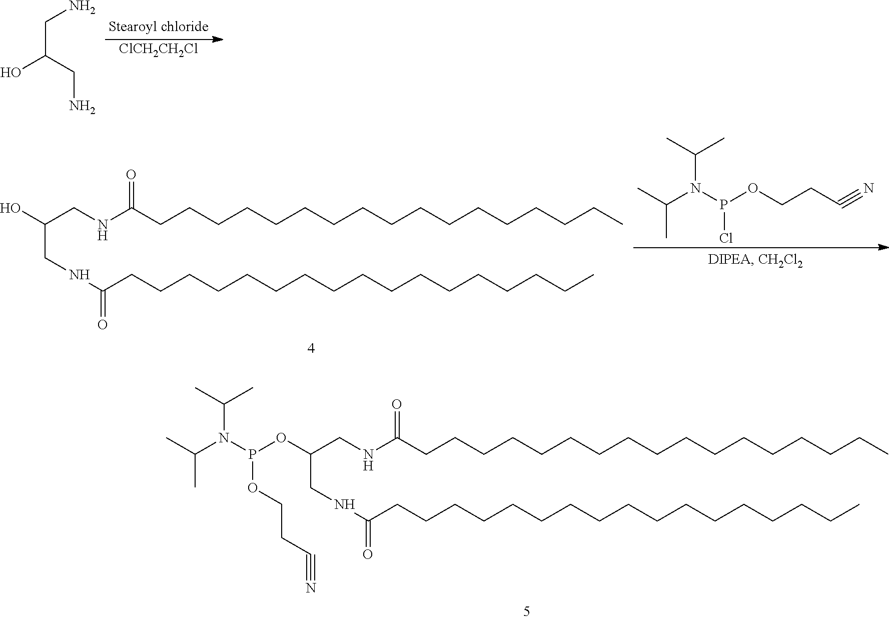

Synthesis of compound 4. A solution of stearoyl chloride (6.789 g, 22.41 mmol) in ClCH2CH2C1 (50 ml) was added dropwise to a solution of 1,3-diamino-2-dydroxypropane (1.0 g, 11.10 mmol) in ClCH2CH2C1 (100 ml) and TEA (2.896 g, 22.41 mmol). The reaction mixture was stirred for 2 hours at room temperature and 70° C. overnight. The solution was then cooled to RT, filtered, and the solid was washed with CH2Cl2, CH3OH, 5% NaHCO3 and ethyl ether. The solid was dried under vacuum to give compound 4 as a white solid (yield: 90%).

Synthesis of compound 5. Compound 4 (5.8 g, 9.31 mmol) was dissolved in anhydrous CH2Cl2 (100 ml), and DIPEA (4.2 ml, 18.62 mmol) was injected. The solution was cooled on an ice bath and 2-Cyanoethyl N,N-diisopropylchlorophosphoramidite (8.6 mL, 0.47 mmol) was added under dry nitrogen. After stirring at RT for 1 hour, the solution was heated to 60° C. for 90 minutes. After cooling to RT, the solution was washed with 5% NaHCO3 and brine, dried over Na2SO4 and concentrated in vacuum. The product was purified by precipitation from CH3CN to afford compound 5 (4 g, 55% yield) as white solids. 31P NMR (CDCl3) 154 ppm.

Synthesis of Aptamer-lipid Sequence. An ABI3400 DNA/RNA synthesizer (Applied Biosystems, Foster City, Calif.) was used for the preparation of all DNA sequences. All oligonucleotides were synthesized based on solid-state phosphoramidite chemistry at a 1 μmmol scale. The aptamer-lipid sequences listed in Table 1 (and shown in FIG. 26) were synthesized in controlled-pore glass columns with a 3′-(6-FAM), TAMR or biotin TEG covalently linked to the CPG substrate. The complete aptamer-lipid sequences were then deprotected in AMA solution (concentrated ammonia hydroxide:methylamine=1:1) at 65° C. for 15 minutes for FAM/biotin labeling or a solution of methnol: tert-butylamine:water (1:1:2) at 65° C. for 3 hours for TMR labeling. A ProStar HPLC (Varian, Walnut Creek, Calif.) with a C8 column (from Alltech, Deerfield, Ill.) was used for probe purification. Deprotected sequences were purified with a linear elution gradient with TEAA (triethylammonium acetate) in acetonitrile changing from 20% to 70% over a 30 min period. The collection from the first HPLC separation was then vacuum dried. Spacer C18 was used as the linker between DNA and lipid tail. Lipid tail phosphoramidite dissolved in methylene chloride was directly coupled onto the sequence by the synthesizer. The synthesis of lipid tail phosphoramidite is described in the Supporting Information section. A Cary Bio-300 UV spectrometer (Varian, Walnut Creek, Calif.) was used to measure absorbance for probe quantification.

Flow Cytometric Analysis. To demonstrate the cell-specific targeting capabilities of aptamer-lipids, fluorescence measurements were made using a FACScan cytometer (Becton Dickinson Immunocytometry Systems, San Jose, Calif.). The binding was performed by the following procedure. 250 nM aptamer-micelle/library-micelle (based on lipid unit concentration) in binding buffer was added to about 1 million cells in the individual flow tubes. The prewarmed mixture was incubated at 37° C. in a cell incubator for various time periods. After incubation, the cells were immediately washed twice with cold washing buffer. The fluorescence was determined by counting 15,000 events.

Competition assays to measure the off rate of the aptamer-cell interaction (Koff) were performed as follows. An aptamer concentration of 250 nM was incubated with cells for 20 minutes at 4° C. for 20 minutes to allow the aptamer to bind to the target in the cell membrane. After the incubation, the cells were washed to remove unbound aptamer. Finally, the labeled cells were incubated with 2.5 uM unlabeled aptamer at 4° C. for various incubation times. The mean fluorescence intensity was measured at different incubation times after a brief vortexing of the cell mixture. Koff was obtained by fitting the dependence of the binding percentage over time to the equation Y=Y0+Aexp (KoffX) where Y is binding percentage and X is time. The fluorescence intensity before the displacement was normalized to 100% binding. Additionally, the equilibrium dissociation constants (Kd) of the aptamer-cell interactions were obtained by fitting the dependence of fluorescence intensity of specific binding on the concentration of the aptamers to the equation Y=BmaxX/(Kd+X) by SigmaPlot (Jandel, San Rafael, Calif.).

Preparation of biotin-TDO5-micelles doped with dyes. Dye-doped aptamer-micelles were prepared by the precipitation and membrane dialysis method. 15 μmol of dry biotin-TDO5-micelle (based on lipid unit) was dissolved in 20 μL ethanol and then thoroughly mixed with 6.74 nmol CellTracker™ Green BODIPY. The solution was then added dropwise into 100 μL deionized water while stirring. After stirring for about 3 hours to evaporate the ethanol, the aqueous solution was dialyzed against 1.5 L water (Spectra/MWCO3500) for 2 days. The water was changed 5 times during the dialysis period. Finally, the solution was filtered through a 0.22 um filter to remove any undesirable aggregates and stored at 4° C.

Confocal Imaging. Cell images were made with a confocal microscope setup consisting of a Leica TCS SP5 Laser Scanning Confocal Microscope. For real-time monitoring, the fluorescent images were taken every minute at a fixed depth. 2 uM cell tracker dye and/or about 4.5 uM aptamer-micelle (based on lipid unit concentration) were incubated with the cells in complete cell medium at 37° C. For the co-localization experiment, cells were incubated with TMR-TDO5-micelle (250 nM) in RPMI-1640 complete medium at 37° C. for 3 hours, and then AF633-transferrin (60 nM) was added 30 minutes before the termination of incubation. Incubation was stopped by placing the cell on ice immediately after washing with cold washing buffer.

Flow Channel Device Preparation and Incubation under Continuous Flow. For micelle-buffer incubation, a PDMS flow channel was used. PDMS devices were fabricated using a process similar to what is described in the literature (178, 198). The layout of the device was designed in AutoCAD and printed on a transparency using a high-resolution printer. The pattern on the transparency was transferred to a silicon wafer via photolithography. The silicon wafer was etched to a depth of 25 μm in a deep reactive ion etching machine. The resulting silicon wafer with the desired pattern served as a mold to fabricate a number of PDMS devices. Sylgard 184 (Corning) reagents were prepared and thoroughly mixed by following the manufacturer's instructions. After being degassed to remove bubbles, the mixture was cast on top of the silicon mold. After being cured at room temperature, the PDMS layer was peeled off the silicon mold. Two wells at both ends of the channel and one well in the middle of the channel were created by punching holes in the PDMS. The PDMS slice was reversibly attached to a clean 50×45 mm cover glass (Fisher) to form a device. Avidin and biotin-aptamer solutions were added to the wells at both channel ends sequentially (sgc8 aptamer for CCRF-CEM on the left side and TDO5 aptamer for TDO5 on the right side) and were sucked through channels by applying a vacuum to the middle well. Then the corresponding cell solutions were put to the corresponding end-wells, sucked slowly through the entire channel by applying a vacuum to one of the end-wells, and incubated at 37° C. for 5 minutes. After washing, the DNA-micelles diluted in binding buffer were continuously flushed through the channel at 300 nL/s for 5 minutes by connecting a Microsyringe pump (World Precision Instruments, Inc.) to the end-well of the channel (dynamic incubation). The channel was ready for confocal imaging after three washing steps. In between experiments, the PDMS devices were cleaned by sequential sonication in 20% bleach with 0.1 M NaCl and then 50:1 water:versaclean (Fisher) at 40° C., followed by rinsing in deionized H2O and drying under N2.

For micelle-blood incubation, a simplified flow channel was made from double glass slides glued together by double-sided tape. Instead of a microsyringe pump, a piece of filter paper was used to suck the solutions through the channel with average flow rate ˜300 nL/s.

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### REFERENCES

Hartgerink, J. D.; Beniash, E.; Stupp, S. I. Science 2001, 294, 1684-1688. (b) Hartgerink, J. D.; Beniash, E.; Stupp, S. I. Proc. Natl. Acad. Sci. USA. 2002, 99, 5133-5238. (c) Lim, Y.; Lee, E.; Lee, M. Angew. Chem. Int. Ed. 2007, 46, 9011-9014. Jeong, J. H.; Park, T. G. Bioconjugate Chem. 2001, 12, 917-923. (b) Gosse, C.; Boutorine, A.; Aujard, I.; Chami, M.; Kononov, A.; Cogne-Laage, E.; Allemand, J.-F.; Li, J.; Jullien, L. J. Phys. Chem. B 2004, 108, 6485-6497. (c) Li, Z.; Zhang, Y.; Fullhart, P.; Mirkin, C. A. Nano lett., 2004, 4, 1055-1058. (d) Safak, M.; Alemdaroglu, F. E.; Li, Y.; Ergen, E.; Herrmann A. Angew. Chem. Int. Ed. 2007, 19, 1499-1505. (e) Dentinger, P. M., Simmons, B. A., Cruz, E., Sprague, M. Langmuir, 2006, 22, 2935.
Chan, Y. M.; Boxer, S. Curr. Opin. Chem. Biol. 2007, 11, 1-7.
Discher, D. E.; Eisenberg, A. Science 2002, 297, 967-973. (b) Allen, C.; Maysinger, D.; Eisenberg, A. Colloids Surf. B 1999, 16, 3-27. (c) Torchilin, V. P.; Lukyanov, A. N.; Gao, Z.; Papahadjopoulos-Sternberg, B. Proc. Natl. Acad. Sci. USA. 2003, 100, 6039-6044.
Park, C.; Lee, I. H.; Song, Y.; Rhue, M.; Kim C. Proc. Natl. Acad. Sci. USA. 2006, 103, 1199-1203. (b) Jones, G.; Vullev, V. I. Org. Lett. 2001, 3, 2457-2460.
Conlon, P., Yang, C. J., Wu, Y., Chen, Y., Martinez, K., Kim, Y., Stevens, N., Marti, A. A., Jockusch, S., Turro, N. J., Tan, W. J. Am. Chem. Soc. 2008, 130, 336-342.
Krishna, M. M. G. J. Phy. Chem. A. 1999, 103, 3589-3595.
Tummino, P. J.; Gafni, A. Biophys. J. 1993, 64, 1580-1587.
Smiddy, M. A.; Martin, J.-E. G. H.; Kelly, A. L.; Kruif, C. G.; Huppertz, T. J. Dairy Sci. 2006, 89, 1906-1914.
Liu, H.; Chen, Y.; He, Y.; Ribbe, A. E.; Mao, C. Angew. Chem. Int. Ed. 2006, 45, 1942-1945.
Liang, X.; Mao, G.; Ng, S. K. Y. Colloids Surf. B 2004, 34, 41-51
Lukyanov, A. N.; Gao, Z.; Mazzola, L.; Torchilin, V. P. Pharm Res. 2002, 19, 1424-1429.
(a) Letsinger, R. L.; Zhang, G.; Sun, D. K.; Ikeuchi, T.; Sarin, P. T. Proc. Natl. Acad. Sci. USA. 1989, 86, 6553-6556. (b) Soutschek, J. et. al.; Nature, 2004, 432, 173-178. (c) Bijsterbosch, M. K.; Rump, E. T.; De Vrueh, R. L. A.; Dorland, R.; Veghel, R.; Tivell, K. L.; Biessen, E. A. L.; Berkel, T. J. C.; Manoharan, M. Nucleic Acids Res. 2000, 28, 2717-2725.
Skobridis, K.; Husken, D.; Nicklin, P.; Haner, R. ARKIVOC, 2005, 459.
Niemeyer, C. M. Angew. Chem. Int. Ed. 2001, 40, 4128-4158.
Yang, J.; Chen, H.; Vlahov, I. R.; Cheng, J.; Low, P. S. Proc. Natl. Acad. Sci. USA. 2006, 103, 13872-13877.
Lu, Y., Liu, J. Curr. Opin. Biotech. 2006, 17, 580-588
Asanuma, H., Liang, X., Nishioka, H., Matsunaga, D.; Liu, M., Kamiyama, M. Synthesis of azobenzene-tethered DNA for reversible photo-regulation of DNA functions: hybridization and transcription. Nature Protocols. 2, 203-212 (2007).
Storhoff, J. J., Elghanian, R., Mucic, R. C., Mirkin, C. A., Letsinger, R1 L. One-pot colorimetric differentiation of polynucleotides with single base imperfections using gold nanoparticle probes. J. Am. Chem. Soc. 120, 1959-1964 (1999).
(20) Gold, L., Janjic, N., Schmidt, P., Vargeese. C. Vascular endothelial growth factor (VEGF) nucleic acid ligand complexes. U.S. Pat. No. 6,168,778.
Bao Q, Shi Y (2007) Apoptosome: a platform for the activation of initiator caspases. Cell Death and Differentiation 14: 56-65.
Mikhail A S, Allen C (2009) Block copolymer micelles for delivery of cancer therapy: Transport at the whole body, tissue and cellular levels. Journal of Control Release 138: 214-223.
Ding K, Alemdaroglu F E, Borsch M, Berger R, Herrmann A (2007) Engineering the structural properties of DNA block copolymer micelles by molecular recognition. Angew Chem Int Ed Engl 46: 1172-1175.
Alemdaroglu F E, Alemdaroglu N C, Langguth, P, Herrmann A (2008) Cellular uptake of DNA block copolymer micelles with different shapes. Macromolecular Rapid Communications 29: 326-329.
Jeong J H, Park T G (2001) Novel polymer-DNA hybrid polymer micelles composed of hydrophobic poly(D,L-lactic-co-glycolic acid) and hydrophilic oligonucleotides. Bioconjugate Chemistry 12: 917-923.
Alemdaroglu F E, Alemdaroglu N C, Langguth P, Herrmann A (2008) DNA block copolymer micelles-A combinatorial tool for cancer nanotechnology. Advanced Materials 20, 899-902.
Tuerk C, Gold L (1990) Systematic evolution of ligands by exponential enrichment-RNA ligands to bacteriophage-T14 DNA-polymerase. Science 249: 505-510.
Ellington A D, Szostak J W (1990) In vitro selection of RNA molecules that bind specific ligands. Nature 346: 818-822.
Shangguan D, et al. (2006) Aptamers evolved from live cells as effective molecular probes for cancer study. Proceedings of the National Academy of Sciences of the United States of America 103: 11838-11843.
Liu J W, Lu Y (2006) Fast colorimetric sensing of adenosine and cocaine based on a general sensor design involving aptamers and nanoparticles. Angewandte Chemie-International Edition 45: 90-94.
Yang H H, Liu H P, Kang H Z, Tan W H. (2008) Engineering target-responsive hydrogels based on aptamer-target interactions. Journal of the American Chemical Society 130: 6320-6321.
Tang Z W, Shangguan D, Wang K M, Shi H, Sefah K, Mallikratchy P, Chen H W, Li Y, Tan W H. (2007) Selection of aptamers formolecular recognition and characterization of cancer cells. Analytical Chemistry 79: 4900-4907.
Mallikaratchy P, Tang Z W, Kwame S, Meng L, Shangguan D H, Tan W H. (2007) Aptamer directly evolved from live cells recognizes membrane bound immunoglobin heavy mu chain in Burkitt's lymphoma cells. Molecular & Cellular Proteomics 6: 2230-2238.
Chen H T, et al. (2008) Release of hydrophobic molecules from polymer micelles into cell membranes revealed by Forster resonance energy transfer imaging. Proceedings of the National Academy of Sciences of the United States of America 105: 6596-6601.
Dautry-Varsat A, Ciechanover A, Lodish H F. (1983) pH and the recycling of transferrin during receptor-mediated endocytosis. Proceedings of the National Academy of Sciences of the United States of America 80: 2258-2262.
Tsui N B Y, Ng E K O, Lo Y M D. (2002) Stability of endogenous and added RNA in blood specimens, serum, and plasma Clinical Chemistry 48: 1647-1653.
Jain R K. (1988) Determinants of tumor blood flow—a review. Cancer Research 48:2641-2658.
Duffy D C, McDonald J C, Schueller O J A, Whitesides G M. (1998) Rapid prototyping of microfluidic systems in poly(dimethylsiloxane). Analytical Chemistry 70: 4974-4984.
Phillips J A, Xu Y, Xia Z, Fan Z H, Tan W H. (2009) Enrichment of cancer cells using aptamers immobilized on a microfluidic channel. Analytical Chemistry 81: 1033-1039.
Yang C J; Wang L; Wu Y; Kim Y; Medley C D.; Lin H; Tan W (2007), Synthesis and investigation of deoxyribonucleic acid/locked nucleic acid chimeric molecular beacons, Nucleic Acids Research, 35(12): 4030-4041.
Shangguan D; Tang Z; Mallikaratchy P; Xiao Z; Tan W (2007) Optimization and modifications of aptamers selected from live cancer cell lines, ChemBioChem, 8: 603-606.
Beckerle, C. M. Cell adhesion, Oxford University Press, 2002.
Ringeisen, B. R.; Othon, C. M.; Barron, J. A.; Young, D.; Spargo, B. J. Biotechnol J. 2006, 1, 930-48.
Jordan, P.; Leach, J.; Padgett, M.; Blackburn, P.; Isaacs, N.; Goksör, M.; Hanstorp, D.; Wright, A.; Girkin, J.; Cooper, J. Lab Chip. 2005, 5, 1224-1228.
Nahmias, Y.; Odde, D. J. Nature Protocols 2006, 1, 2288-2296.
Chiou, P. Y.; Ohta, A. T.; Wu, M. C. Nature, 436, 370-372.
Mirkin, C. A.; Letsinger, R. L.; Mucic, R. C.; Storhoff, J. J. Nature, 1996, 382, 607-609.
Alivisatos, A. P.; Johnsson, K. P.; Peng, X.; Wilson, T. E.; Loweth, C. J.; Bruchez, M. P.; Schultz, P. G. Nature, 1996, 382, 609-611.
Chen, Y.; Liu, H.; Ye, T.; Kim, J.; Mao, C. J. Am. Chem. Soc. 2007, 129, 8696-8697.
Yoshina-Ishii C.; Boxer, S. G.; J. Am. Chem. Soc., 2003, 125, 3696-3697.
Jakobsen, U.; Simonsen, A. C.; Vogel, S. J. Am. Chem. Soc. 2008, 130, 10462-10463.
Stengel, G.; Zahn, R.; Höök, F. J. Am. Chem. Soc. 2007, 129, 9584-9585.
Yang, H.; Liu, H.; Kang, H.; Tan, W. J. Am. Chem. Soc. 2008, 130, 6320-6321.
Lin, C.; Katilius, E.; Liu, Y.; Zhang, J.; Yan, H. Angew. Chem. Int. Ed. 2006, 45, 5296-5301.
Chandra, R. A.; Douglas, E. S.; Mathies, R. A.; Bertozzi, C. R.; Francis, M. B. Angew. Chem. Int. Ed. 2006, 45, 896-901.
Borisenko, G. G.; Zaitseva, M. A.; Chvilin, A. N.; Pozmogova, G. E. Nucleic Acids Res. 2009, 37, e28.
Gartner, Z.; Bertozzi, C. R. Proc. Natl. Acad. Sci. USA. 2009, 106, 4606-4610.
Liu, H.; Zhu, Z.; Kang, H.; Wu, Y.; Sefah, K.; Tan, W. Chem. Eur. J. accepted.
Shea, R. G. Marsters, J. C.; Bischofberger, N. Nucleic Acids Res. 1990, 18, 3777-3783.
Chan, Y. M.; Lengerich, B. V.; Boxer, S. G. Biointerphases 2008, 3, FA17-FA21.
Bunge, A.; Kurz, A.; Windeck, A. K.; Korte, T.; Flasche, W.; Liebscher, J.; Herrmann, A.; Huster, D. Langmuir 2007, 23, 4455-4464.
Shangguan, D., Li, Y., Tang, Z., Cao, Z. C., Chen, H. W., Mallikaratchy, P., Sefah, K., Yang, C. J., and Tan, W. Proc. Natl. Acad. Sci. U.S.A 2006, 103, 11838-11843.
Tang, Z., Shangguan, D., Wang, K., Shi, H., Sefah, K., Mallikaratchy, P., Chen, H. W., Li, Y., and Tan, W. Anal. Chem. 2007. 79, 4900-4907.
Pfeiffer, I.; Höök, F. J. Am. Chem. Soc. 2004, 126, 10224-10225.
Liu, H.; Zhu, Z.; Kang, H.; Wu, Y.; Sefah, K.; Tan, W. Chemistry, a European Journal, accepted.
Gold, L., Janjic, N., Schmidt, P., Vargeese. C. Vascular endothelial growth factor (VEGF) nucleic acid ligand complexes. U.S. Pat. No. 6,168,778, 2001.

1. A population of micelles comprising:
oligonucleotides; and hydrophobic lipid elements, wherein the population is homogenous.

2. The population of micelles of claim 1, wherein the population is homogenous in diameter and weight.

3. (canceled)

4. The population of micelles of claim 1, wherein the hydrophobic lipid elements form a hydrophobic core.

5. The population of micelles of claim 1, wherein the oligonucleotides comprise single stranded DNA.

6-9. (canceled)

10. The population of micelles of claim 1, wherein the oligonucleotides and hydrophobic elements are linked by a covalent bond.

11. The population of micelles of claim 10, wherein the covalent bond is a cleavable or non-cleavable linkage.

12. (canceled)

13. The population of micelles of claim 1, further comprising reporter molecules.

14. (canceled)

15. The population of micelles of claim 13, wherein the reporter molecules are pyrene molecules.

16-19. (canceled)

20. The population of micelles of claim 4, wherein the oligonucleotide is selected from the group consisting of: an antisense oligonucleotide, a decoy oligonucleotide, a siRNA, a DNAzyme, a ribozyme, and an aptamer.

21. (canceled)

22. The population of micelles of claim 20, wherein the aptamer is selected from the group consisting of: SEQ ID NO: 1 (AAC ACC GGG AGG ATA GTT CGG TGG CTG TTC AGG GTC TCC TCC CGG TGA), SEQ ID NO: 2 (ATC CAG AGT GAC GCA GCA GAT CAG TCT ATC TTC TCC TGA TGG GTT CCT AGT TAT AGG TGA AGC TGG ACA CGG TGG CTT AGT), SEQ ID NO: 3 (ACA GCA GAT CAG TCT ATC TTC TCC TGA TGG GTT CCT ATT TAT AGG TGA AGC TGT, SEQ ID NO: 4 (AAC ACC GGG AGG ATA GTT CGG TGG CTG TTC AGG GTC TCC TCC CGG TGA TTT TTT TTT TTT TTT), SEQ ID NO: 5(5′-AAC ACC GGG AGG ATA GTT CGG TGG CTG TTC AGG GTC TCC TCC CGG TGA TTT TTT TTT T-biotin-3), SEQ ID NO: 6 (5′-ATC TAA CTG CTG CGC CGC CGG GAA AAT ACT GTA CGG TTA GAT TTT TTT TTT-biotin-3)′; or
wherein the micelle is selected from the sequence corresponding to SEQ ID NO: 8 (5′-lipid tail-(CH2CH2O)24-AAC ACC GGG AGG ATA GTT CGG TGG CTG TTC AGG GTC TCC TCC CGG TGA-FAM-3′, SEQ ID NO: 9 (5′-lipid tail-(CH2CH2O)24-AAC ACC GGG AGG ATA GTT CGG TGG CTG TTC AGG GTC TCC TCC CGG TGA-biotin-3′, SEQ ID NO: 10 (5′-lipid tail-(CH2CH2O)24-AAC ACC GGG AGG ATA GTT CGG TGG CTG TTC AGG GTC TCC TCC CGG TGA-TMR-3′), SEQ ID NO: 11 (5′-lipid tail-(CH2CH2O)24-ATC CAG AGT GAC GCA GCA GAT CAG TCT ATC TTC TCC TGA TGG GTT CCT AGT TAT AGG TGA AGC TGG ACA CGG TGG CTT AGT-FAM-3′), SEQ ID NO: 12 (5′-lipid tail-(CH2CH2O)24-ACA GCA GAT CAG TCT ATC TTC TCC TGA TGG GTT CCT ATT TAT AGG TGA AGC TGT-FAM-3′, SEQ ID NO: 13(5′-lipid tail-(CH2CH20)24-(N)n*-FAM-3′.

23-26. (canceled)

27. The population of micelles of claim 4, further comprising an agent loaded into the hydrophobic core.

28. (canceled)

29. The population of micelles of claim 27, wherein the agent is a therapeutic agent or an imaging agent.

30. (canceled)

31. The population of micelles of claim 29, wherein the therapeutic agent is selected from the group consisting of: a drug, a toxin, a gene, a small molecule and an oligonucleotide.

32. The population of micelles of claim 31, wherein the oligonucleotide is selected from the group consisting of: an antisense oligonucleotide, a decoy oligonucleotide, a siRNA, a DNAzyme, a ribozyme, and an aptamer.

33-37. (canceled)

38. A pharmaceutical composition comprising a population of micelles comprising: oligonucleotides; and hydrophobic lipid elements, wherein the population is homogenous, and a pharmaceutically acceptable carrier.

39-69. (canceled)

70. A method of preparing a homogenous population of micelles comprising:
preparing oligonucleotides;
preparing hydrophobic lipid elements; and
mixing oligonucleotides and hydrophobic lipid elements in an aqueous medium, thereby forming a homogenous population of micelles.

71-73. (canceled)

74. A method of preparing a homogenous population of micelles comprising:
preparing oligonucleotides;
preparing hydrophobic lipid elements;
preparing pyrene molecules; and
mixing oligonucleotides, hydrophobic lipid elements and pyrene molecules in an aqueous medium, thereby forming a homogenous population of micelles.

75-86. (canceled)

87. A method of delivering an oligonucleotide to a target cell comprising contacting the target cell with a population of micelles of claim 1.

88-90. (canceled)

91. The method of claim 87, wherein the oligonucleotide is selected from the group consisting of: an antisense oligonucleotide, a decoy oligonucleotide, a siRNA, a DNAzyme, a ribozyme, and an aptamer.

92-95. (canceled)

96. A method for treating a disease or disorder in a subject comprising administering to the subject the population of micelles of claim 1.

97. A method of delivering an oligonucleotide to a target cell comprising:
contacting the target cell with a population of micelles comprising oligonucleotides and hydrophobic lipid elements; and
incubating the target cell with the population of micelles;
thereby delivering an oligonucleotide to the target cell.

98-101. (canceled)

102. A method for treating a disease or disorder in a subject comprising:
administering to the subject a population of micelles comprising oligonucleotides and hydrophobic lipid elements, thereby treating the disease or disorder in the subject.

103-115. (canceled)

116. A kit comprising:
oligonucleotides; and hydrophobic lipid elements, and instructions for use.

117-119. (canceled)
